(19) United States
(12) Patent Application Publication
(10) Pub. No.: US 2009/0111743 A1
(43) Pub. Date: Apr. 30, 2009

(54) Cysteine-branched heparin-binding growth factor analogs

(75) Inventor: Kazuyuki Takahashi, Germantown, MD (US)

Correspondence Address: PEACOCK MYERS, P.C., 201 THIRD STREET, N.W., SUITE 1340, ALBUQUERQUE, NM, 87102, US

(73) Assignee: BioSurface Engineering Technologies, Inc., Rockville, MD (US)

(21) Appl. No.: 11/362,137

(22) Filed: Feb. 23, 2006

Related U.S. Application Data
(60) Provisional application No. 60/656,713, filed on Feb. 25, 2005.

(51) Int. Cl. A61K 38/08; C07K 7/00; C07K 14/00; A61K 38/16; A61K 38/10; A61K 38/00

(52) U.S. Cl. 514/12; 530/330; 530/329; 530/328; 530/327; 530/326; 530/325; 530/324; 514/17; 514/16; 514/15; 514/14; 514/13

(57) ABSTRACT

The present invention provides a heparin binding growth factor analog of any of formula I-VIII and methods and uses thereof.

### CROSS-REFERENCE TO RELATED APPLICATIONS

This application claims priority to and the benefit of the filing of U.S. Provisional Patent Application Ser. No. 60/656,713,570 entitled “Cysteine-Branched Heparin-Binding Growth Factor Analogs” filed on Feb. 22, 2005 and the specification and claims thereof are incorporated herein by reference.

### INTRODUCTION

The invention relates to the field of synthetic peptides and analogs of heparin-binding growth factors, including homodimeric and heterodimeric chain synthetic heparin-binding growth factor analogs wherein a linear homodimeric or heterodimeric sequence is covalently bonded to a heparin-binding sequence by means of a side chain in the homodimeric or heterodimeric sequence. The invention further relates to the clinical uses of such analogs as soluble drugs and as coatings for medical devices.

### BACKGROUND OF THE INVENTION

Note that the following discussion refers to a number of publications by author(s) and year of publication. Discussion of such publications herein is given for more complete background and is not to be construed as an admission that such publications are prior art for patentability determination purposes.

The heparin-binding growth factors (HBGFs) constitute a large class of growth factors that includes the 23 fibroblast growth factors identified to date (FGFs 1-23), HBBM (heparin-binding brain mitogen), HB-GAF (heparin-binding growth associated factor), HB-EGF (heparin-binding EGF-like factor) HB-GAM (heparin-binding growth associated molecule), TGF-α (transforming growth factor-α), TGF-βs (transforming growth factor-βs), PDGF (platelet-derived growth factor), EGF (epidermal growth factor), VEGF (vascular endothelial growth factor), IGF-1 (insulin-like growth factor-1), IGF-2 (insulin-like growth factor-2), HGF (hepatocyte growth factor), IL-1 (interleukin-1), IL-2 (interleukin-2), IFN-α (interferon-α), IFN-γ (interferon-γ), TNF-α (tumor necrosis factor-α), SDGF (Schwannoma-derived growth factor) and the many other growth factors, cytokines, lymphokines and chemokines that have an affinity for heparin.

Peptides from natural HBGFs that bind heparin-binding growth factor receptors have been identified. See for example Ray et al., Proc. Natl. Acad. Sci. USA 94:7047-7052 (1997). These authors demonstrated that two amino acid sequences from FGF-2 are sufficient to block the mitogenic activity of FGF-2 on neural progenitor cells. The first peptide is a ten amino acid sequence, from amino acids 65-74, the second peptide extends from amino acids 115-129.

In an alternative approach, an artificial peptide that binds a heparin-binding growth factor receptor (HBGFR) was identified by a phage display method. Ballinger et al., Nature BioTechnology 17:1199-1204 (1999) used this technique to isolate a 28 amino acid peptide called C19, binds FGF-2 receptors, but by itself fails to stimulate biological activity. The peptide has no amino acid sequence identity with any known FGF.

HBGFs useful in prevention or therapy of a wide range of diseases and disorders may be purified from natural sources or produced by recombinant DNA methods, however, such preparations are expensive and generally difficult to prepare.

Some efforts have been made to generate heparin-binding growth factor analogs. For example, natural PDGF occurs as an A chain and a B chain arranged in head-to-head (AA or BB) homodimers, or (AB or BA) heterodimers. Thus, U.S. Pat. No. 6,350,731 to Jehanli et al. discloses PDGF analogs in which two synthetic PDGF receptor-binding domains are covalently linked through a polyglycine or an N-(4-carboxy-cyclohexylmethyl)-maleimide (SMCC) chain to mimic the natural active polypeptide dimer.

U.S. Pat. No. 6,235,716 to Ben-Sasson discloses analogs of angiogenic factors. The analogs are branched multivalent ligands that include two or more angiogenic homology regions connected by a multilinker backbone.

U.S. Pat. No. 5,770,704 (the '704 patent) to Godowski discloses conjugates for activating receptor tyrosine kinases, cytokine receptors and members of the nerve growth factor receptor superfamily. The conjugates include at least two ligands capable of binding to the cognate receptor, so that the binding of the respective ligands induces oligomerization of these receptors. The ligands disclosed in the '704 patent are linked by covalent attachment to various nonproteinaceous polymers, particularly hydrophilic polymers, such as polyvinylalcohol and polyvinylpyrrolidone, and the polyvinylalkene ethers, including polyethylene glycol and polypropylene glycol. The ligands include hepatocyte growth factor (HGF) peptide variants that each bind HGF receptor, thereby causing receptor dimerization and activation of the biological activity of the HGF receptor dimer.

U.S. Pat. No. 6,284,503 (the '503 patent) to Caldwell et al. discloses a composition and method for regulating the adhesion of cells and biomolecules to hydrophobic surfaces and hydrophobic coated surfaces for cell adhesion, cell growth, cell sorting and biological assays. The composition is a biomolecule conjugated to a reactive end group activated polymer. The end group activated polymer includes a block copolymer surfactant backbone and an activation or reactive group. The block copolymer may be any surfactant having a hydrophobic region capable of adsorbing onto a hydrophobic surface, and a hydrophilic region which extends away from the surface when the hydrophobic region is adsorbed onto the hydrophobic surface. The '503 patent discloses that the biomolecules that may be conjugated to the end group activated polymer include natural or recombinant growth factors, such as PDGF, EGF, TGFα, TGFβ, NGF, IGF-I, IGF-II, GH and GHRF, as well as multi-CSF (II-3), GM-CSF, G-CSF, and M-CSF.

Other workers have described compositions that include homologs and analogs of fibroblast growth factors (FGFs). See for example U.S. Pat. No. 5,679,673 to Lappi and Baird; U.S. Pat. No. 5,989,866 to Deisher et al. and U.S. Pat. No. 6,294,359 to Fiddes et al. These disclosures relate to FGF homologs or analogs that are either conjugated to a toxic moiety and are targeted to the FGF receptor-bearing cells; or are homologs or analogs that modulate the biological pathways through the signal transduced by the FGF receptor upon binding by the FGF homolog or analog.

A series of patent applications to Kochendoerfer et al. disclose polymer-modified proteins, including synthetic chemokines and erythropoiesis stimulating proteins. See, for example, International Publications WO 02/04105, WO 02/19963 and WO 02/20033. These include chemically ligated peptide segments of a polypeptide chain of a synthetic erythropoiesis protein, such that a polypeptide chain results, with a water soluble polymer attached at one or more glycosylation sites on the protein. These applications also disclose synthetic chemokines, which are also polymer modified, and are asserted to be antagonists. However, heparin-binding domains are not disclosed. Other erythropoietin mimetics are known, such as those disclosed in U.S. Pat. Nos. 5,773,569 and 5,830,851 to Wrighton et al.

A series of applications with some inventors in common, including U.S. patent application Ser. No. 10/644,703, entitled Synthetic Heparin-Binding Growth Factor Analogs, filed on Aug. 19, 2003, and U.S. patent application Ser. No. 10/224,268, entitled Synthetic Heparin-Binding Growth Factor Analogs, filed on Aug. 20, 2002, disclose constructs in which two receptor-binding domains are branched from an amine of a backbone amino acid through a peptide bond.

The above described homologs, analogs, conjugates or ligands each include a receptor-binding domain. However, none of the disclosed compositions further include both a linker, providing for the linking of at least two receptor-binding domains through sulfur complexation.

International Publication WO 00/18921 to Ballinger and Kavanaugh discloses a composition consisting of fusion proteins having FGF receptor affinity linked to an “oligomerization domain”, either directly or through a linking group. The oligomerization domain ranges in length from about 20 to 300 residues, and includes constructs such as transcription factors, Fc portions of IgG, leucine zippers and the like. The oligomerization domains disclosed are homodimeric domains, wherein a single FGF receptor affinity fusion protein is linked to a single domain, such as a leucine zipper, which in turn is linked to a similar molecule by means of cysteine residues at both the amino and carboxy termini of the leucine zippers, such that two parallel leucine zippers, each with a single FGF receptor affinity fusion protein, are cross-linked by means of disulfide bonds. It is also disclosed that fusion proteins may include a heparin binding domain, such as the use of jun as a multimerization domain, which is asserted to be a heparin binding domain. Thus the compositions disclosed by Ballinger and Kavanaugh are all composed of a single receptor-binding sequence covalently attached to an oligomerization domain, whereby two or more similar oligomerization domains, each with a single receptor-binding sequence, are conjoined by means of either an association provided by the oligomerization domain, or alternatively, are chemically cross-linked to provide for the covalent bonding of the individual components.

The above described homologs, analogs, conjugates or ligands each include a receptor-binding domain. However, none of the disclosed compositions further include both a linker, providing for the linking of at least two receptor-binding domains to the linker through a side chain of the receptor-binding domains, and further providing a single non-signaling peptide containing a heparin-binding domain. Moreover, none of these or other known heparin-binding growth factor analogs provide the advantages described herein below. There is still a need for new peptide analogs of HBGFs, particularly for those that function as agonists, and preferably those that contain two receptor-binding domains specific for a HBGFR. In particular, there is still a need for cost-effective synthetic peptide agonists of heparin-binding growth factor receptors, particularly synthetic heparin-binding growth factor agonists useful for coating medical devices and as soluble biologics, and as pharmaceutical agents for treating a variety of conditions.

### SUMMARY OF THE INVENTION

One aspect of the present invention provides a heparin-binding growth factor analog of formula I:

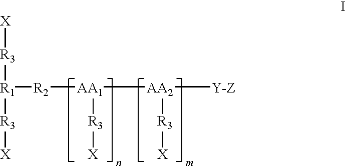

wherein:

each X a peptide chain that (i) has a minimum of three amino acid residues, (ii) has a maximum of about fifty amino acid residues, and (iii) binds a heparin-binding growth factor receptor (HBGFR);

R₁ is a trifunctional amino acid residue covalently bonded to one X through the N terminus amine of R₁ and the remaining X through the C terminus carboxyl of R₁;

R₂ is a linker comprising a chain from 3 to about 20 atoms covalently bonded to the side chain of R₁ and Y when n=0, or to the side chain of R₁ and to the N terminus amine of AA₁ when n=1;

Each R₃ is independently from 0 to about 3 amino acid residues which are the same or different;

Y is a linker comprising a chain from 0 to about 50 atoms covalently bonded to R₂ and Z when n=0, or to AA₁ and Z when n=1 and m=0, or to AA₂ and Z when n=1 and m=1;

Z is a non-signaling peptide chain that includes a heparin binding domain, comprising an amino acid sequence that comprises (i) a minimum of one heparin binding motif, (ii) a maximum of about ten heparin binding motifs, and (iii) a maximum of about thirty amino acids;

AA₁ and AA₂ are each independently a trifunctional amino acid residue, wherein X is covalently bonded through the side chain of AA₁ and AA₂; and

n is 0 or 1, and when n is 1, m is 0 or 1, and when n is 0, m is 0.

Another aspect of the present invention provides a heparin-binding growth factor analog of formula II:

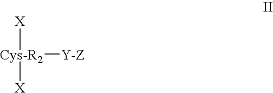

wherein:
Cys is cysteine, and R₂ is a linker consisting of a sulfhydryl reactive homo-bifunctional cross-linker and a second Cys or comprising a hetero-bifunctional cross-linker. All other features are as indicated for formula I.

One aspect of the present invention provides a heparin-binding growth factor analog of formula III:

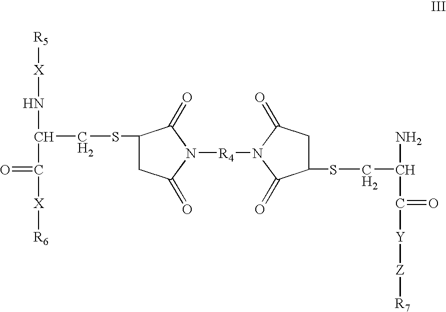

wherein:
R₄ is a linker comprising a chain of between 1 and about 10 backbone atoms selected from carbon, oxygen, sulfur and nitrogen or mixtures thereof;

R₅ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative;

R₆ is OH, NH₂, or NH—R₅; and

R₇ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative. All other features are as indicated for formula I.

Still another aspect of the present invention provides a heparin-binding growth factor analog of formula IV:

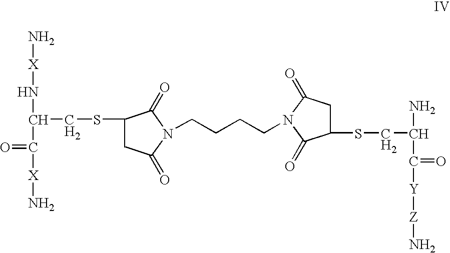

wherein:
X is any of SEQ ID NOS:6-55; Y is between two and about five amino acid residues selected from the group consisting of 6-aminohexanoic acid, 7-aminoheptanoic acid, 9-aminononanoic acid and mixtures thereof, and Z is any of SEQ ID NOS:2-5 or SEQ ID NO:58.

Yet another aspect of the present invention provides a heparin-binding growth factor analog of formula I-IV wherein the covalent bonds between the side chain of R₁ and R₂ and between R₂ and Z when n=0, or between AA₁ and Z when n=1 and m=0, or between AA₂ and Z when n=1 and m=1, comprise an amide, disulfide, thioether, Schiff base, reduced Schiff base, imide, secondary amine, carbonyl, urea, hydrazone or oxime bond.

One aspect of the present invention provides a heparin-binding growth factor analog of formula I-IV wherein the side chains of AA₁ and AA₂ comprise reactive carboxyl groups.

Yet another aspect of the present invention provides a heparin-binding growth factor analog of formula I-IV wherein the side chain of R₁ comprises a reactive sulfhydryl group.

One aspect of the present invention provides a heparin-binding growth factor analog of formula I-IV wherein R₁ is an L- or D-3-mercapto amino acid.

Still another aspect of the present invention provides a heparin-binding growth factor analog of formula I-IV wherein the L- or D-3-mercapto amino acid is L- or D-cysteine, L- or D-penicillamine, 3-mercapto phenylalanine, or a derivative of any of the foregoing.

One aspect of the present invention provides a heparin-binding growth factor analog of formula I-IV wherein X is any of SEQ ID NO:5 to SEQ ID NO:55 and Z is RKRLDRIAR (SEQ ID NO:58), RKRKLERIAR (SEQ ID NO:2) RKRKLGRIAR (SEQ ID NO:3) or RKRKLWRARA (SEQ ID NO:4).

Yet another aspect of the present invention provides a heparin-binding growth factor analog of formula I-IV wherein one or more of R₃ is between one and about three amino acid residues selected from the group consisting of glycine, 6-aminohexanoic acid, 7-aminoheptanoic acid, 9-aminononanoic acid and mixtures thereof.

One aspect of the present invention provides a heparin-binding growth factor analog of formula I-IV wherein X comprises an amino acid sequence found in any of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-I (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, glial derived neurotrophic factor (GDNF), GRO2, hepatoma-derived growth factor (HDGF), and granulocyte-macrophage colony stimulating factor (GMCSF), a homolog of any amino acid sequence found in the foregoing, a reverse sequence of any amino acid sequence found in the foregoing, or a homolog of a reverse sequence of any amino acid residue found in the foregoing.

Another aspect of the present invention provides a heparin-binding growth factor analog of formula I-IV wherein Y comprises between one and about thirty-three ethylene glycol units.

Still another aspect of the present invention provides a heparin-binding growth factor analog of formulas I-IV wherein Y comprises a branched or unbranched, saturated or unsaturated alkyl chain of between one and about twenty carbon atoms.

One aspect of the present invention provides a heparin-binding growth factor analog of formulas I-IV wherein Y comprises [NH₂—(CH₂)ₚCO]_{q} wherein p is from 1 to about 10 and q is from 1 to about 20.

Another aspect of the present invention provides a heparin-binding growth factor analog of formulas I-IV wherein Y comprises a peptide sequence comprising from one to about 16 Gly residues.

One aspect of the present invention provides a heparin-binding growth factor analog of formula I-IV wherein each heparin binding motif of Z is BxBB or BBBxxB, wherein each B is independently lysine, arginine, ornithine, or histidine, and each x is a independently a naturally occurring amino acid. In another aspect, Z of the heparin-binding growth factor analog may comprise at least two heparin-binding motifs.

Still another aspect of the present invention provides a pharmaceutical composition comprising the heparin-binding growth factor analog of formulas I-IV or a pharmaceutically acceptable salt thereof and a pharmaceutical carrier.

One aspect of the present invention provides a heparin-binding growth factor analog of any of formulas I-IV wherein X and Z are synthetic peptide chains.

Yet another aspect of the present invention provides a heparin-binding growth factor analog of any of formulas I-IV having a linker that (i) is hydrophobic, (ii) comprises a chain of a minimum of about 9 and a maximum of about 50 atoms, and (iii) is not found in the natural ligand of the heparin-binding growth factor receptor (HBGFR) which X binds.

Still another aspect of the present invention provides a heparin-binding growth factor analog of any of formulas I-IV having an avidity for heparin such that the synthetic heparin-binding growth factor analog binds heparin in 0.15 M NaCl, but is eluted by 1 M NaCl.

One aspect of the present invention provides a heparin-binding growth factor analog of formula V:

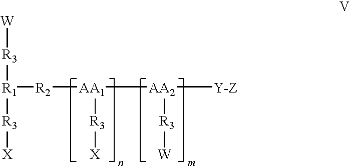

wherein:

each X and each W is a peptide chain differing by at least one amino acid residue that (i) has a minimum of three amino acid residues, (ii) has a maximum of about fifty amino acid residues, and (iii) binds a heparin-binding growth factor receptor (HBGFR);

R₁ is a trifunctional amino acid residue covalently bonded to one X through the N terminus amine of R₁ and the remaining X through the C terminus carboxyl of R₁;

R₂ is a linker comprising a chain from 3 to about 20 atoms covalently bonded to the side chain of R₁ and Y when n=0, or to the side chain of R₁ and to the N terminus amine of AA₁ when n=1;

Each R₃ is independently from 0 to about 3 amino acid residues which are the same or different;

Y is a linker comprising a chain from 0 to about 50 atoms covalently bonded to R₂ and Z when n=0, or to AA₁ and Z when n=1 and m=0, or to AA₂ and Z when n=1 and m=1;

Z is a non-signaling peptide chain that includes a heparin binding domain, comprising an amino acid sequence that comprises (i) a minimum of one heparin binding motif, (ii) a maximum of about ten heparin binding motifs, and (iii) a maximum of about thirty amino acids;

AA₁ and AA₂ are each independently a trifunctional amino acid residue, wherein X and W are covalently bonded through the side chain of AA₁ and AA₂, respectively; and,

n is 0 or 1, and when n is 1, m is 0 or 1, and when n is 0, m is 0.

Yet another aspect of the present invention provides a heparin-binding growth factor analog of formula V wherein X, W and Z are synthetic peptide chains.

One aspect of the present invention provides a heparin-binding growth factor analog of formula V wherein Y further comprises a linker that (i) is hydrophobic, (ii) comprises a chain of a minimum of about 9 and a maximum of about 50 atoms, and (iii) is not found in the natural ligand of the heparin-binding growth factor receptor (HBGFR) which X binds.

Still another aspect of the present invention provides a heparin-binding growth factor analog of formula V wherein the heparin-binding growth factor analog has an avidity for heparin such that the synthetic heparin-binding growth factor analog binds heparin in 0.15 M NaCl, but is eluted by 1 M NaCl.

Yet still another aspect of the present invention provides a heparin-binding growth factor analog of formula VI:

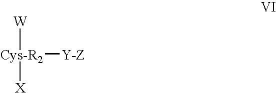

wherein:
Cys is cysteine; and
R₂ is a linker consisting of a sulfhydryl reactive homo-bifunctional cross-linker and a second Cys or comprising a hetero-bifunctional cross-linker. All other features are as represented for formula V.

One aspect of the present invention provides a heparin-binding growth factor analog of formula VII:

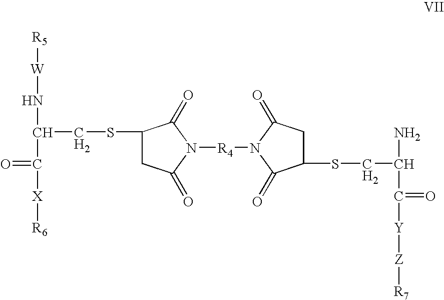

wherein:

R₄ is a linker comprising a chain of between 1 and about 10 backbone atoms selected from carbon, oxygen, sulfur and nitrogen or mixtures thereof;

R₅ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative;

R₆ is OH, NH₂, or NH—R₅; and

R₇ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative. All other features are as presented for formula V.

Yet another aspect of the present invention provides a heparin-binding growth factor analog of formula VIII:

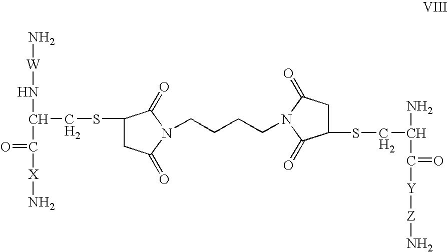

wherein:
X and W are each independently selected from any of SEQ ID NOS:6-55;
Y is between two and about five amino acid residues selected from the group consisting of 6-aminohexanoic acid, 7-aminoheptanoic acid, 9-aminononanoic acid and mixtures thereof; and Z is any of SEQ ID NOS:2-5 or SEQ ID NO:58. All other features are as represented for formula V.

Still another aspect of the present invention provides a heparin-binding growth factor analog of formula V-VIII wherein the covalent bonds between the side chain of R₁ and R₂ and between R₂ and Z when n=0, or between AA₁ and Z when n=1 and m=0, or between AA₂ and Z when n=1 and m=1, comprise an amide, disulfide, thioether, Schiff base, reduced Schiff base, imide, secondary amine, carbonyl, urea, hydrazone or oxime bond.

Another aspect of the present invention provides a heparin-binding growth factor analog of formula I-IV wherein X and W each independently comprise an amino acid sequence found in any of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, glial derived neurotrophic factor (GDNF), GRO2, hepatoma-derived growth factor (HDGF), and granulocyte-macrophage colony stimulating factor (GMCSF), a homolog of any amino acid sequence found in the foregoing, a reverse sequence of any amino acid sequence found in the foregoing, or a homolog of a reverse sequence of any amino acid residue found in the foregoing.

Other aspects, objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

Additional objects and advantages of the present invention will be apparent in the following detailed description read in conjunction with the accompanying drawing figures.

### DETAILED DESCRIPTION OF THE INVENTION

In one embodiment, each synthetic HBGF analog of the invention contains two identical sequences that are analogs of a particular HBGF that binds to a HBGFR, or alternatively each synthetic HBGF analog of the invention contains two sequences, both of which bind to a HBGFR. The homodimeric sequences may be derived from any portion of a HBGF. The synthetic HBGF analog may be an analog of a hormone, a cytokine, a lymphokine, a chemokine or an interleukin, and may bind to any HBGFR for any of the foregoing.

In one aspect the synthetic HBGF analog of the present invention is a molecule of any one of formulas I to IV. HBGFs include any growth factor that binds selectively to heparin. For example, the HBGF can be any of the known FGFs (FGF-1 to FGF-23), HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, or glial derived neurotrophic factor (GDNF), GRO2, hepatoma-derived growth factor (HDGF), granulocyte-macrophage colony stimulating factor (GMCSF), and the many growth factors, cytokines, interleukins and chemokines that have an affinity for heparin. It is also contemplated that agents of the invention can be modified through the introduction of appropriate binding sequences to direct analogs of growth factors, cytokines, interleukins, and chemokines, which do not normally bind to heparin, to have heparin-binding affinity.

In another aspect the synthetic HBGF analog of the present invention is a molecule of any one of formulas V to VIII, which is a heterodimeric construct including at least one X region and one W region, wherein the X and W regions vary by at least one residue. In a preferred embodiment, each of the X and W regions are different sequences derived from different regions of the same growth factor. X and W regions may be derived in that they are identical to or homologous with a sequence within a growth factor. It is to be understood that any definition of X contained herein is also equally applicable to W, and W is not hereafter defined.

The amino acid sequences of many of these and other HBGFs are available from the National Library of Medicine Protein Database at the internet site accessible through the world wide web address ncbi.nlm.nih.gov/entrez. These HBGF amino acid sequences on the foregoing internet site are hereby incorporated by reference. The use of synthetic HBGF analogs incorporating the amino acid sequences of the receptor binding domains from these and other HBGFs is specifically contemplated in the present invention.

In particular embodiments of the present invention, the synthetic HBGF analog of the present invention consists essentially of the molecule of any one of formulas I to VIII, i.e. the molecule of any one of formula I to VIII is the major active component in the synthetic HBGF analog composition.

### The Heparin-Binding Growth Factors of Formulas I to IV

The regions X and Z of the synthetic HBGF analogs of formulas I to IV include amino acid residues, and optionally the region Y includes amino acid residues. An amino acid residue is defined as —NHRCO—, where R can be hydrogen or any organic group. The amino acids can be D-amino acids or L-amino acids. Additionally, the amino acids can be α-amino acids, β-amino acids, γ-amino acids, or δ-amino acids and so on, depending on the length of the carbon chain of the amino acid.

The amino acids of the X, Y and Z component regions of the synthetic HBGF analogs of the invention can include any of the twenty amino acids found naturally in proteins, i.e. alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine, (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

Furthermore, the amino acids of the X, Y and Z component regions of the synthetic HBGF analogs of the invention can include any of the naturally occurring amino acids not found naturally in proteins, e.g. β-alanine, betaine (N,N,N-trimethylglycine), homoserine, homocysteine, γ-amino butyric acid, ornithine, and citrulline.

Additionally, the amino acids of the X, Y and Z component regions of the synthetic HBGF analogs of the invention can include any of the non-biological amino acids, i.e. those not normally found in living systems, such as for instance, a straight chain amino-carboxylic acid not found in nature. Examples of straight chain amino-carboxylic acids not found in nature include 6-aminohexanoic acid, 7-aminoheptanoic acid, 9-aminononanoic acid and the like.

In formula I, two X regions are covalently linked to R₁, either directly or through an R₃ group, where R₁ is a trifunctional amino acid residue, preferably a trifunctional alpha amino acid residue. It is to be appreciated that such covalent bonds may be to any chemically permitted functional group. Where the trifunctional amino acid residue is an amino acid with a reactive sulfhydryl side chain, such as cysteine, it is possible and contemplated that one X is covalently bonded through the N-terminus amine group, the remaining X is covalently bonded through the C-terminus carboxyl group, and the cysteine is bound to R₂ through the reactive sulfhydryl side chain. Similarly, one X may be covalently bonded to an R₃ group, such as between one and three glycines, which R₃ group is covalently bonded through the C-terminus carboxyl group of R₁ and the other X is similarly bonded through the N-terminus amine group of R₁ through an R₃ group, again such as one to three glycines. Similar approaches may be employed with other trifunctional amino acid residues, using cross-linkers as hereafter described.

The amino acids AA₁ and AA₂ can be any trifunctional amino acid residue, preferably a trifunctional alpha amino acid residue. In one preferred embodiment, the trifunctional amino acid residue is a diamine amino acid, such as for instance lysine or ornithine, or any other amino acid having two amino groups.

The term “homologous”, as used herein refers to peptides that differ in amino acid sequence at one or more amino acid positions when the sequences are aligned. For example, the amino acid sequences of two homologous peptides can differ only by one amino acid residue within the aligned amino acid sequences of five to ten amino acids. Alternatively, two homologous peptides of ten to fifteen amino acids can differ by no more than two amino acid residues when aligned. In another alternative, two homologous peptides of fifteen to twenty or more amino acids can differ by up to three amino acid residues when aligned. For longer peptides, homologous peptides can differ by up to approximately 5%, 10%, 20% or 25% of the amino acid residues when the amino acid sequences of the two peptide homologs are aligned.

Particularly useful amino acid sequences as X regions of formulas I to IV include homologs of fragments of naturally occurring HBGFs that differ from the amino acid sequences of natural growth factor in only one or two or a very few positions. Such sequences preferably include conservative changes, where the original amino acid is replaced with an amino acid of a similar character according to well known principles; for example, the replacement of a non-polar amino acid such as alanine with valine, leucine, isoleucine or proline; or the substitution of one acidic or basic amino acid with another amino acid of the same acidic or basic character.

In another alternative, the X regions of the synthetic HBGF analog can include an amino acid sequence that shows no detectable homology to the amino acid sequence of any HBGF. Peptides or growth factor analogs useful as components of the X region of the synthetic analogs of the present invention, that have little or no amino acid sequence homology with the cognate growth factor and yet bind HBGFRs may be obtained by any of a wide range of methods, including for instance, selection by phage display. See as an example: Sidhu et al. Phage display for selection of novel binding peptides. Methods Enzymol. 328:333-63 (2000).

The X region of the synthetic HBGF analogs of the invention can have any length that includes an amino acid sequence that effectively binds an HBGFR. Preferably, the X regions of the synthetic HBGF analogs have a minimum length of at least approximately three amino acid residues. More preferably, the X regions of the synthetic HBGF analogs have a minimum length of at least approximately six amino acid residues. Most preferably the X regions of the synthetic HBGF analogs have a minimum length of at least approximately ten amino acid residues. The X regions of the synthetic HBGF analogs of the invention preferably also have a maximum length of up to approximately fifty amino acid residues, more preferably a maximum length of up to approximately forty amino acid residues, and most preferably a maximum length of up to approximately thirty amino acid residues.

The R₂ regions of formulas I or II can include a chain of atoms or a combination of atoms that form a chain. Typically, the chains are chains of carbon atoms, that may also optionally include oxygen, nitrogen or sulfur atoms, such as for example chains of atoms formed from amino acids (e.g. amino acids found in proteins, as listed above; naturally occurring amino acids not found in proteins, such as ornithine and citrulline; or non natural amino acids, such as amino hexanoic acid; or a combination of any of the foregoing amino acids). It is also contemplated that agents such as polyethylene glycol (PEG), polyethylene oxide (PEO), amino polyethylene glycol, bis-amine-PEG, and other variants of polyethylene glycol known to those skilled in the art can similarly be used.

The chain of atoms of the R₂ region of formula I and IV is covalently attached to R₁ and AA₁ if n=1, or to R₁ and Y if n=0. The covalent bonds can be, for example, peptide, amide, thioether or ester bonds. Preferably, the R₂ region includes a chain of a minimum of about three atoms. For example, where the covalent bonds are peptide bonds, the R₂ region may be formed from a chain of at least one, at least two or at least three amino acids. However, where other than peptide bonds are employed, the R₂ region may further include a cross-linking moiety. For example, in formula II the R₂ region is a linker consisting of a sulfhydryl reactive homo-bifunctional cross linker and a second Cys, or alternatively includes a hetero-bifunctional cross-linker.

In one preferred embodiment, two X regions form a single linear peptide construct, joined by an R₁ group that is a trifunctional amino acid residue, and optional one or two R₃ groups. The trifunctional amino acid residue may, for example, have a reactive sulfhydryl group in the side chain, such as an L- or D-3-mercapto amino acid, including but not limited to L- or D-cysteine, L- or D-penicillamine, 3-mercapto phenylalanine, or a derivative of any of the foregoing. The R₁ trifunctional amino acid residue is covalently bonded to the X regions by peptide bonds, such that the single linear peptide construct is, by way of example only, X—C—X or X—R₃—C—R₃—X, where C is L- or D-cysteine, and each X is covalently linked to C by peptide bonds, directly in the case of X—C—X, and indirectly in the case of X—R₃—C—R₃—X. Similarly, the R₂ group can include a trifunctional amino acid residue with a reactive sulfhydryl group in the side chain, again covalently bonded to the Y region by an ordinary peptide bond. In one generalized description, this includes the following general formula:

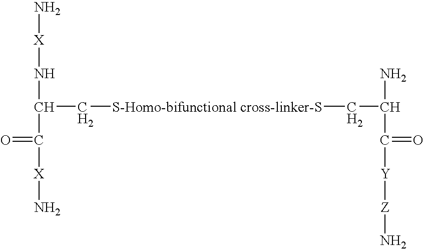

or alternatively:

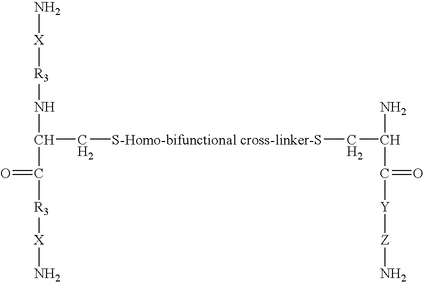

In these formulas, the “homo-bifunctional cross-linker” forms a part of R₂, together with the C residue to which Y is covalently bonded. Any sulfhydryl reactive homo-bifunctional crosslinking agent may be employed, such as for example a maleimide cross-linker, a haloacetyl cross-linker or a pyridyl disulfide cross-linker. A large number of such sulfhydryl cross-linkers, such as maleimide cross-linkers, are known.

For example, in maleimide cross-linkers of the general formula:

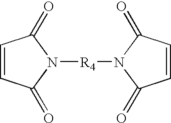

R₄ may be a C₁ to C₈ alkyl chain, such as for example 1,2-bis-maleimidoethane, 1,4-bis-malimidobutane or 1,6-bis-maleimidohexane, or may be an aryl group such as phenyl, such as for example 1,4-phenylene dimaleimide or 1,2-phenylene dimaleimide, or may be an aliphatic chain containing one or more oxygen (O), sulfur (S) or nitrogen (N) chain members, and optionally a ketone, such as for example dithio-bis-maleimidoethane, maleimidopropionic acid maleimidomethyl ester, bis-maleimidomethylether, 1,11-bis-maleimido-(PEO)₄, 1,8-bis-maleimido-(PEO)₃, and so on.

In yet another embodiment, any of a number of homo- or hetero-functional electrophilically-activated PEGs may be employed, including those that contain functional groups such as succinimidyl propionate, succinimidyl butanoate, N-hydroxysuccinimide, benzotriazol carbonate, aldehydes, acetaldehyde diethyl acetal, or vinylsulfone, and others known to those skilled in the art.

In yet another embodiment, a hetero-bifunctional cross-linker is employed. Hetero-bifunctional reagents which cross-link by two different coupling moieties can be particularly useful. Thus, the coupling moiety on R₁ is a cysteine residue and, on either Y or a part of R₂, a residue or other moiety with an amino group, such that a cross-linker for an amino group and sulfhydryl group is employed, for example m-maleimidobenzoyl-N-hydroxysuccinimide ester. Alternatively the cross-linker reagent links two amino groups, for example N-5-azido-2-nitrobenzoyloxysuccinimide, an amino group and a carboxyl group, for example 4-[p-azidosalicylamido]butylamine, or an amino group and a guanadium group that is present in the side chain of arginine, for example p-azidophenyl glyoxal monohydrate.

Preferably, the R2 region includes a chain of a maximum of about twenty backbone atoms. The amino acid sequence of the R₂ region, if amino acid residues are employed therein, is preferably an artificial sequence, i.e. it does not include any amino acid sequence of four or more amino acid residues found in a natural ligand of a HBGF.

In the synthetic HBGF analogs of the present invention, in one preferred embodiment the Y region of any of formulas I to IV is a linker that is sufficiently hydrophobic to non-covalently bind the HBGF analog to a polystyrene or polycaprolactone surface, or the like. In addition, the Y region may bind to other hydrophobic surfaces, particularly the hydrophobic surfaces formed from materials used in medical devices. Such surfaces are typically hydrophobic surfaces. Examples of suitable surfaces include but are not limited to those formed from hydrophobic polymers such as polycarbonate, polyester, polypropylene, polyethylene, polystyrene, polytetrafluoroethylene, expanded polytetrafluoroethylene, polyvinyl chloride, polyamide, polyacrylate, polyurethane, polyvinyl alcohol, polyurethane, poly ethyl vinyl acetate, poly(butyl methacrylate), poly(ethylene-co-vinyl acetate), polycaprolactone, polylactide, polyglycolide and copolymers of any two or more of the foregoing; siloxanes such as 2,4,6,8-tetramethylcyclotetrasiloxane; natural and artificial rubbers; glass; and metals including stainless steel, titanium, platinum, and nitinol. Preferably, the binding of the HBGF analogs to the hydrophobic surface is of sufficient quantity to be detected by an analytical method such as an enzyme-linked immunoassay or a biological assay.

According to one embodiment of the invention, the Y region of formulas I to IV includes a chain of atoms or a combination of atoms that form a chain. Typically, the chains are chains of carbon atoms, that may also optionally include oxygen, nitrogen or sulfur atoms, such as for example chains of atoms formed from amino acids (e.g. amino acids found in proteins, as listed above; naturally occurring amino acids not found in proteins, such as ornithine and citrulline; or non natural amino acids, such as amino hexanoic acid; or a combination of any of the foregoing amino acids).

The chain of atoms of the Y region of formula I to IV is covalently attached to either R₂ or, if provided, AA₁ or AA₂, and to peptide Z. The covalent bonds can be, for example, peptide, amide, thioether or ester bonds. Preferably, the Y region includes a chain of a minimum of about nine atoms. More preferably, the Y region includes a chain of a minimum of about twelve atoms. Most preferably, the Y region includes a chain of a minimum of about fifteen atoms. For example, the Y region may be formed from a chain of at least four, at least five or at least six amino acids. Alternatively, the Y region may be formed from a chain of at least one, at least two, or at least three aminohexanoic acid residues.

Preferably, the Y region includes a chain of a maximum of about fifty atoms. More preferably, the Y region includes a chain of a maximum of about forty-five atoms. Most preferably, the Y region includes a chain of a maximum of about thirty-five atoms. For example, the Y region may be formed from a chain of up to about twelve, up to about fifteen, or up to about seventeen amino acids.

The amino acid sequence of the Y region is preferably an artificial sequence, i.e. it does not include any amino acid sequence of four or more amino acid residues found in a natural ligand of a HBGF.

In a particular embodiment, the Y region includes a hydrophobic amino acid residue, or a chain of hydrophobic amino acid residues. The Y region can, for example, include one or more aminohexanoic acid residues, such as one, two, three or more aminohexanoic acid residues. Alternatively, the Y region can include up to about twelve, up to about fifteen, or up to about seventeen ethylene glycol residues. In another alternative embodiment, the Y region can include a combination of amino acid hydrophobic residues.

In another particular embodiment, the Y region of the molecule can include a branched or unbranched, saturated or unsaturated alkyl chain of between one and about twenty carbon atoms. In a further embodiment, the Y region can include a chain of hydrophilic residues, such as for instance, ethylene glycol residues. For instance, the Y region can include at least about three, or at least about four, or at least about five ethylene glycol residues.

The Z region of the molecule of any of formulas I to IV is a heparin-binding region and can include one or more heparin-binding motifs, BBxB or BBBxxB as described by Verrecchio et al. J. Biol. Chem. 275:7701 (2000). Alternatively, the Z region can include both BBxB and BBBxxB motifs (where B represents lysine, arginine, or histidine, and x represents a naturally occurring, or a non-naturally occurring amino acid). For example, the heparin-binding motifs may be represented by the sequence [KR][KR][KR]X(2)[KR] (SEQ ID NO:1), designating the first three amino acids as each independently selected from lysine or arginine, followed by any two amino acids and a sixth amino acid which is lysine or arginine.

The number of heparin binding motifs is variable. For instance, the Z region may include at least one, at least two, at least three or at least five heparin-binding motifs. Where there are more than one heparin-binding motifs, the motifs may be the same or different. Alternatively, the Z region includes up to a maximum of about ten heparin-binding motifs. In another alternative embodiment, the Z region includes at least four, at least six or at least eight amino acid residues. Further, in certain embodiments the Z region includes up to about twenty, up to about, twenty-five, or up to about thirty amino acid residues. It is to be realized that, in part, the avidity of the Z region for heparin is determined by the particular heparin-binding motifs selected and the number of such motifs in Z. Thus for particular applications both the selection and number of such motifs may be varied to provide optimal heparin binding of the Z region.

In a preferred embodiment, the amino acid sequence of the Z region is RKRKLERIAR (SEQ ID NO:2). In another embodiment, the amino acid sequence of the Z region is RKRKLGRIAR (SEQ ID NO:3). In yet another embodiment, the amino acid sequence of the Z region is RKRKLWRARA (SEQ ID NO:4). In yet another embodiment, the amino acid sequence of the Z region is RKRKLERIARC (SEQ ID NO:5). The presence of a terminal cysteine residue optionally affords the opportunity to link other molecules, including detection reagents such as fluorochromes, radioisotopes and other detectable markers, to the Z region, as well as the opportunity to link toxins, immunogens and the like.

Heparin-binding domains that bear little or no sequence homology to known heparin-binding domains are also contemplated in the present invention. As used herein the term “heparin-binding” means binding to the —NHSO₃⁻ and sulfate modified polysaccharide, heparin, and also binding to the related modified polysaccharide, heparan. Such domains are contemplated to exhibit binding in physiological solutions including 0.15 M NaCl, and are expected to uncomplex at salt concentrations greater than 0.5 M NaCl.

The Z region of the synthetic HBGF analogs of the present invention confers the property of binding to heparin in low salt concentrations, up to about 0.15 M NaCl, optionally up to about 0.48 M NaCl, forming a complex between heparin and the Z region of the factor analog. The complex can be dissociated in 1 M NaCl to release the synthetic HBGF analog from the heparin complex.

The Z region is a non-signaling peptide. Accordingly, when used alone the Z region binds to heparin which can be bound to a receptor of a HBGF, but the binding of the Z region peptide alone does not initiate or block signaling by the receptor.

The C-terminus of the Z region may be blocked or free. For example, the C terminus of the Z region may be the free carboxyl group of the terminal amino acid, or alternatively, the C terminus of the Z region may be a blocked carboxyl group, such as for instance, an amide group.

### The Heparin-Binding Growth Factors of Formulas V to VIII

The regions X and W regions of the synthetic HBGF analogs of formulas V to VIII are as defined above for each X region. All other assigned values and descriptions, including without limitation cross-linkers, Y, and Z, are as defined for the heparin-binding growth factors of formulas I to IV.

### DEFINITIONS

As used here and elsewhere, the following terms have the meanings given.

The term “alkene” includes unsaturated hydrocarbons that contain one or more double carbon-carbon bonds. Examples of such alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length in either a straight or branched configuration. Examples of such alkyl radicals include methyl, ethyl, propyl, isopropyl, butyl, sec-butyl, tertiary butyl, pentyl, isopentyl, hexyl, isohexyl, and the like.

The term “aryl” includes a monovalent or bicyclic aromatic hydrocarbon radical of 6 to 12 ring atoms, and optionally substituted independently with one or more substituents selected from alkyl, haloalkyl, cycloalkyl, alkoxy, alkythio, halo, nitro, acyl, cyano, amino, monosubstituted amino, disubstituted amino, hydroxy, carboxy, or alkoxy-carbonyl. Examples of an aryl group include phenyl, biphenyl, naphthyl, 1-naphthyl, and 2-naphthyl, derivatives thereof, and the like.

The term “aralkyl” includes a radical —R^{a}R^{b} where R^{a} is an alkylene (a bivalent alkyl) group and R^{b} is an aryl group as defined above. Examples of aralkyl groups include benzyl, phenylethyl, 3-(3-chlorophenyl)-2-methylpentyl, and the like. The term “aliphatic” includes compounds with hydrocarbon chains, such as for example alkanes, alkenes, alkynes, and derivatives thereof.

The term “acyl” includes a group RCO—, where R is an organic group. An example is the acetyl group CH₃CO—.

A peptide or aliphatic moiety is “acylated” when an alkyl or substituted alkyl group as defined above is bonded through one or more carbonyl {—(C═O)—} groups. A peptide is most usually acylated at the N-terminus.

An “amide” includes compounds that have a trivalent nitrogen attached to a carbonyl group (—CO.NH₂).

An “amine” includes compounds that contain an amino group (—NH₂).

A “diamine amino acid” is an amino acid or residue containing two reactive amine groups and a reactive carboxyl group. Representative examples include 2,3 diamino propionyl amino acid residue, 2,4 diamino butylic amino acid residue, lysine or ornithine.

A “trifunctional amino acid” is an amino acid or residue with three reactive groups, one the N-terminus amine, a second the C-terminus carboxyl, and the third comprising all or a part of the side chain. Trifunctional amino acids thus include, by way of example only, diamine amino acids; amino acids with a reactive sulfhydryl group in the side chain, such as mercapto amino acids including cysteine, penicillamine, or 3-mercapto phenylalanine; amino acids with a reactive carboxyl group in the side chain, such as aspartic acid and glutamic acid; and amino acids with a reactive guanadium group in the side chain, such as arginine.

### FGF Synthetic Analogs

In another particular aspect, the invention provides a synthetic FGF peptide analog. The synthetic FGF analogs represented by any of formulas I to IV above, wherein X is an FGF analog which can be any FGF, such as any of the known FGFs, including all 23 FGFs from FGF-1 to FGF-23.

The X region of the molecule of formulas I to IV can include an amino acid sequences found in an FGF, such as for instance FGF-2 or FGF-7. Alternatively, the X regions can include sequences not found in the natural ligand of the FGFR bound by the molecule.

The Y region of the synthetic FGF peptide analogs of any of formulas I to IV are not necessarily hydrophobic, and thus, if present, can be polar, basic, acidic, hydrophilic or hydrophobic. Thus, the amino acid residues of the Y region of synthetic FGF peptide analogs can include any amino acid, or polar, ionic, hydrophobic or hydrophilic group.

The X region of synthetic FGF peptide analogs can include an amino acid sequence that is 100% identical to an amino acid sequence found in a fibroblast growth factor or an amino acid sequence homologous to the amino acid sequence of a fibroblast growth factor. For instance, the X region can include an amino acid sequence that is at least about 50%, at least about 75%, or at least about 90% homologous to an amino acid sequence from a fibroblast growth factor. The fibroblast growth factor can be any fibroblast growth factor, including any of the known or yet to be identified fibroblast growth factors.

In a particular embodiment, the synthetic FGF analog of the invention is an agonist of the HBGFR. When bound to the HBGFR, the synthetic HBGF analog initiates a signal by the HBGFR.

In a further particular embodiment, the synthetic FGF analog of the invention is an antagonist of the HBGFR. When bound to the HBGFR, the synthetic HBGF analog blocks signaling by the HBGFR.

In another particular embodiment of the present invention, the synthetic FGF analog is an analog of FGF-2 (also known as basic FGF, or bFGF). In another particular embodiment of the present invention, the binding of the synthetic FGF analog to an FGF receptor initiates a signal by the FGF receptor. In a further particular embodiment, the binding of the synthetic FGF analog to the FGF receptor blocks signaling by the FGF receptor.

In a yet further particular embodiment, the present invention provides a synthetic FGF analog of FGF-2. In another particular embodiment, the present invention provides a synthetic FGF analog of FGF-2, wherein the amino acid sequence of the X region is YRSRKYTSWYVALKR (SEQ ID NO:6) from FGF-2. In yet another particular embodiment, the present invention provides a synthetic FGF analog wherein the amino acid sequence of the X region is NRFHSWDCIKTWASDTFVLVCYDDGSEA (SEQ ID NO:7). In yet another particular embodiment, the present invention provides a synthetic FGF-2 analog wherein the amino acid sequence of the X region is HIKLQLQAEERGVVS (SEQ ID NO:8).

In a yet further particular embodiment, the invention provides a synthetic FGF analog of FGF-1, wherein the X region is YISKKHAEKNWFVGLKK (SEQ ID NO:9). This sequence is derived from amino acids bridging the beta 9 and beta 10 loop of FGF-1. In yet another particular embodiment, an FGF-1 analog is provided wherein the X region is HIQLQLSAESVGEVY (SEQ ID NO:10), corresponding to amino acids derived from the β-4 and β-5 region of FGF-1.

In a yet further particular embodiment, the invention provides a synthetic FGF analog of FGF-7, wherein the X region is YASAKWTHNGGEMFVALNQK (SEQ ID NO:11). In yet another embodiment of a synthetic FGF analog of FGF-7, the X region is the amino acid sequence YNIMEIRTVAVGIVA (SEQ ID NO:12).

Other FGF receptor binding domains, derived largely from targeting sequences in the C-terminus of human FGF, include the following sequences shown in Table 1:

TABLE 1

| | PREFERRED X RECEPTOR |
|---|---|
| CYTOKINE | BINDING DOMAIN |
| FGF-10 | YASFNWQHNGRQMYVALNQK |
| | (SEQ ID NO:13) |
| FGF-22 | YASQRWRRRGQPNLALDRR |
| | (SEQ ID NO:14) |
| FGF-9 | YSSNLYKHVDTGRRYYVALNK |
| | (SEQ ID NO:15) |
| FGF-16 | YASTLYKHSDSERQYVALNK |
| | (SEQ ID NO:16) |
| FGF-20 | YSSNIYKHGDTGRRFVALNK |
| | (SEQ ID NO:17) |
| FGF-4 | YESYKYPGMFIALSKN |
| | (SEQ ID NO:18) |
| FGF-6 | YESDLYQGTYILSKYGR |
| | (SEQ ID NO:19) |
| FGF-12 | YSSTLYRQQESGRAWFLGNK |
| | (SEQ ID NO:20) |
| FGF-14 | YSSMLYRQQESGRAWFLGLNK |
| | (SEQ ID NO:21) |
| FGF-13 | YSSMIYRQQQSGRGWYLGLNK |
| | (SEQ ID NO:22) |
| FGF-11 | YASALYRQRRSGRAWYLDK |
| | (SEQ ID NO:23) |

### VEGF Synthetic Analogs

In another particular aspect, the invention provides a synthetic VEGF peptide analog. The synthetic VEGF analogs represented include, in one embodiment, a VEGF analog wherein the amino acid sequence of the X region is APMAEGGGQNHHEVVKFMDV (SEQ ID NO:24). In another embodiment, there is provided a synthetic VEGF peptide analog wherein the amino acid sequence of the X region is GATWLPPNPTK (SEQ ID NO:25). In yet another embodiment, there is provided a synthetic VEGF peptide analog wherein the amino acid sequence of the X region is NFLLSWVHWSLALLLYLHHA (SEQ ID NO:26).

### BMP Synthetic Analogs

In another particular aspect, the invention provides a synthetic BMP peptide analog. The synthetic bone morphogenic protein analogs include embodiments wherein the X region includes the amino acid sequence LYVDFSDVGWNDW (SEQ ID NO:27), AISMLYLDENEKVVL (SEQ ID NO:28), ISMLYLDENEKVVLKNY (SEQ ID NO:29), EKVVLKNYQDMVVEG (SEQ ID NO:30), LVVKENEDLYLMSIAC (SEQ ID NO:31), AFYCHGECPFPLADHL (SEQ ID NO:32), or PFPLADHLNSTNHAIVQTLVNSV (SEQ ID NO:33).

Alternatively, in another particular aspect the invention provides synthetic BMP, TGF or GDF (growth differentiation factor) peptide analogs as shown in Table 2 wherein the transforming growth factor family member peptides are particularly useful in augmenting the activity of endogenous or artificial BMP peptides or TGF peptides, wherein is shown (under the heading “preferred receptor binding domain”) the sequence forming all or part of the X region of constructs of any of formulas I to IV.

TABLE 2

| | PREFERRED X RECEPTOR |
|---|---|
| CYTOKINE | BINDING DOMAIN |
| TGF-β1 | IVYYVGRKPKVEQLSNMIVRS |
| | (SEQ ID NO:34) |
| TGF-β2 | TILYYIGKTPKIEQLSNMIVKS |
| | (SEQ ID NO:35) |
| TGF-β3 | LTILYYVGRTPKVEQLSNMVV |
| | (SEQ ID NO:36) |
| BMP-2 | AISMLYLDENEKVVLKNYQDMVV |
| | (SEQ ID NO:37) |
| BMP-3 | SSLSILFFDENKNVVLKVYPNMTV |
| | (SEQ ID NO:38) |
| BMP-3β | NSLGVLFLDENRNVVLKVYPNMSV |
| | (SEQ ID NO:39) |
| BMP-4 | AISMLYLDEYDKVVLKNYQEMVV |
| | (SEQ ID NO:40) |
| BMP-5 | AISVLYFDDSSNVILKKYRNMVV |
| | (SEQ ID NO:41) |
| BMP-6 | AISVLYFDDNSNVILKKYRNMVV |
| | (SEQ ID NO:42) |
| BMP-7 | AISVLYFDDSSNVILKKYRNMVV |
| | (:43) |
| BMP-8 | ATSVLYYDSSNNVILRKARNMVV |
| | (SEQ ID NO:44) |
| BMP-9 | ISVLYKDDMGVPTLKYHYEGMSV |
| | (SEQ ID NO:45) |
| BMP-10 | ISILYLDKGVVTYKFKYEGMAV |
| | (SEQ ID NO:46) |
| BMP-11 | INMLYFNDKQQIIYGKIPGMVV |
| | (SEQ ID NO:47) |
| BMP-12 | ISILYIDAANNVVYKQYEDMVV |
| | (SEQ ID NO:48) |
| BMP-13 | ISILYIDAGNNVVYKQYEDMVV |
| | (SEQ ID NO:49) |
| BMP-14 | ISILFIDSANNVVYKQYEDMVV |
| | (SEQ ID NO:50) |
| BMP-15 | ISVLMIEANGSILYKEYEGMIA |
| | (SEQ ID NO:51) |
| GDF-1 | ISVLFFDNSDNVVLRQYEDMVV |
| | (SEQ ID NO:52) |
| GDF-3 | ISMLYQDNNDNVILRHYEDMVV |
| | (SEQ ID NO:53) |
| GDF-8 | INMYLFNGKEQIIYGKIPAMVV |
| | (SEQ ID NO:54) |
| GDF-9 | LSVLTIEPDGSIAYKEYEDMIA |
| | (SEQ ID NO:55) |

### Methods of Synthesizing the Heparin-Binding Growth Factor Analogs

The synthesis of the analogs of the invention can be achieved by any of a variety of chemical methods well known in the art. Such methods include bench scale solid phase synthesis and automated peptide synthesis in any one of the many commercially available peptide synthesizers. Preferably, the synthesizer has a per cycle coupling efficiency of greater than 99 percent.

The analogs of the present invention can be produced by stepwise synthesis or by synthesis of a series of fragments that can be coupled by similar well known techniques. See, for instance, Nyfeler, Peptide synthesis via fragment condensation. Methods Mol. Biol. 35:303-16 (1994); and Merrifield, Concept and early development of solid-phase peptide synthesis. Methods in Enzymol. 289:3-13 (1997). These methods are routinely used for the preparation of individual peptides. It is possible to assemble the analogs of the present invention in component parts, such as peptides constituting the X, Y and Z components thereof, and to thereafter couple such component parts to assemble the analog. See, for instance, Dawson and Kent, Synthesis of native proteins by chemical ligation. Annu. Rev. Biochem. 69:923-960 (2000); and Eom et al., Tandem ligation of multipartite peptides with cell-permeable activity. J. Am. Chem. Soc. 125:73-82 2003).

Peptide libraries that can be used to screen for a desired property, such as binding to an HBGFR, can be prepared by adaptations of these methods. See for instance, Fox, Multiple peptide synthesis, Mol. Biotechnol. 3:249-58 (1995); and Wade and Tregear, Solid phase peptide synthesis: recent advances and applications. Austral. Biotechnol. 3:332-6 (1993).

In a particular embodiment, the synthetic HBGF analog of the invention is an agonist of the HBGFR. When bound to the HBGFR, the synthetic HBGF analog initiates a signal by the HBGFR.

In another particular embodiment, the synthetic HBGF analog of the invention is an antagonist of the HBGFR. When bound to the HBGFR, the synthetic HBGF analog blocks signaling by the HBGFR.

In a particular aspect, the invention provides a method for stimulating growth factor receptor signaling in a cell by contacting the cell with an effective amount of a synthetic HBGF analog according to formulas I to IV. The effective amount can be readily determined by one of skill in the art. The signaling can result in cytokine release from the cell, stimulation or inhibition of proliferation or differentiation of the cell, chemotaxis of the cell, stimulation or inhibition of the immune system of the mammal.

### Methods of Use of the HBGFs of the Invention

The HBGF analogs of the invention provide a cost effective and potentially unlimited source of biologically active molecules that are useful in a number of ways, including as soluble prophylactic or therapeutic pharmaceutical agents, such as for instance for administration as a soluble drug for prevention or treatment of various diseases, including for example, uses in cancer therapy and radioprotection.

The synthetic HBGF analogs of present invention are also useful as biologically active agents for coating of medical devices, such as for instance, sutures, implants and medical instruments to promote biological responses, for instance, to stimulate growth and proliferation of cells, or healing of wounds.

In one aspect, the present invention provides a method and compositions for treating a mammal that has been exposed to a harmful dose of radiation. The method includes administering an effective dose of a synthetic HBGF analog of the invention which is an FGF analog to the mammal. The treatment is particularly useful in the prevention or treatment of mucositis, gastrointestinal syndrome (G.I. syndrome), or radionecrosis such as can result from exposure to radiation. The HBGF analog can be administered parenterally, orally, or topically. Alternatively, the HBGF analog can be delivered loco-regionally, e.g. on an analog coated medical device. In a related embodiment, the present invention provides a method for treating a mammal that has been administered a dose of a chemotherapeutic agent, to ameliorate the toxicity of the chemotherapeutic agent to the mammal. In a particular embodiment of the above-described methods, the mammal is a human. In another particular embodiment of the method, the HBGF analog is an FGF-2 analog or an FGF-7 analog.

In another aspect, the invention provides a method and compositions for treating a mammal with bone injury, by providing a HBGF analog of the present invention having an X region reactive with a BMP HBGFR, such as an analog of BMP-2. For example, such HBGF analogs of the present invention may be administered as a pharmaceutical agent, or may be employed as an additive to bone matrix or bone graft materials.

In another aspect, the invention provides a method and compositions for preparation of cell or organ implant sites. In one embodiment, a homodimeric HBGF analog of FGF-2 of the present invention is administered by a percutaneous route to stimulate localized angiogenesis prior to implant of insulin-secreting pancreatic cells, and thereby improve the survival of the implanted cells. Similarly, a homodimeric HBGF analog of FGF-2 of the present invention is administered into ischemic heart tissue prior to the implant of myocte stem cells.

In another aspect, the invention provides a method and compositions to increase cellular attachment to and cellular retention on blood-contacting surfaces of medical devices. In one embodiment, a homodimeric HBGF analog of VEGF of the present invention is applied on vascular graft materials such that the bound analog recruits and binds circulating endothelial stem cells from the blood, thereby resulting in endothelialization of the graft surface with resultant long-term thromboresistance being imparted to the graft.

In another aspect, the invention provides a method and compositions to increase and provide for membrane-guided tissue growth.

In another aspect, the invention provides a method and composition for treatment of difficult-to-treat dermal wounds, including ulcers. In one embodiment, a homodimeric HBGF analog of TGF-β1 is applied topically in a pharmaceutically acceptable cream or gel for treatment of ulcerated bed sores and similar difficult-to-treat dermal wounds.

In yet another aspect, the invention provides a method and compositions to selectively increase cellular populations in vitro. For example, a homodimeric HBGF analog of TGF-β1 is formulated in a tissue culture medium to specifically stimulate the growth of chondrocytes, stem cells which give rise to chondrocytes, or pluripotent cells which give rise of chondrocytes. Similarly, a homodimeric HBGF analog of VEGF may be employed to stimulate the growth of endothelial cells.

The term “medical device” as used herein means a device that has one or more surfaces in contact with an organ, tissue, blood or other bodily fluid in an organism, preferably a mammal, particularly, a human. Medical devices include, for example, extracorporeal devices for use in surgery such as blood oxygenators, blood pumps, blood sensors, tubing used to carry blood, and the like which contact blood that is returned to the patient. The term can also include endoprostheses implanted in blood contact in a human or animal body, such as vascular grafts, stents, pacemaker leads, heart valves, and the like that are implanted in blood vessels or in the heart. The term can further include devices for temporary intravascular use such as catheters, guide wires, and the like that are placed in blood vessels or the heart for purposes of monitoring or repair. The term can further include nerve electrodes, muscle electrodes, implantable pulse generators, implantable drug pumps, and defibrillators. Moreover, the term medical device can include sutures, graft materials, wound coverings, nerve guides, bone wax, aneurysm coils, embolization particles, microbeads, dental implants, bone prostheses, tissue scaffolds, artificial joints or a controlled release drug delivery devices.

The surface of the medical device can be formed from any of the commonly used materials suitable for use in medical devices, such as for instance, stainless steel, titanium, platinum, tungsten, ceramics, polyurethane, polytetrafluoroethylene, extended polytetrafluoroethylene, polycarbonate, polyester, polypropylene, polyethylene, polystyrene, polyvinyl chloride, polyamide, polyacrylate, polyurethane, polyvinyl alcohol, polycaprolactone, polylactide, polyglycolide, polysiloxanes (such as 2,4,6,8-tetramethylcyclotetrasiloxane), natural rubbers, or artificial rubbers, or block polymers or copolymers thereof.

Methods for coating biological molecules onto the surfaces of medical devices are known. See for instance U.S. Pat. No. 5,866,113 to Hendriks et al., the specification of which is hereby incorporated by reference. Tsang et al. in U.S. Pat. No. 5,955,588 teach a non-thrombogenic coating composition and methods for using the same on medical devices, and is incorporated herein by reference. Zamora et al. in U.S. Pat. No. 6,342,591 teach an amphipathic coating for medical devices for modulating cellular adhesion composition, and is incorporated herein by reference.

In one embodiment, the invention provides a method for delivering an active peptide to a mammal, the method includes (i) providing a medical device coated on its surface with a synthetic HBGF analog of formulas I to IV, the synthetic HBGF analog being bound to the surface of the medical device by non-covalent bonds; and (ii) placing the medical device onto a surface of, or implanting the medical device into, the mammal.

In a particular embodiment of the above method, the non-covalent bonds are associations between the heparin binding domain of the synthetic HBGF analog and a heparin-containing compound bound to the surface of the medical device. The heparin-containing compound bound to the surface of the medical device can be any heparin-containing compound, such as for instance, benzyl-bis(dimethylsilylmethyl)oxy carbamoyl-heparin.

In another particular embodiment of the above method, the medical device is not pre-coated with a heparin-containing compound before being coated with the synthetic HBGF analog of formulas I to IV.

### Heparin-Binding Growth Factor Analog Pharmaceutical Applications

The HBGF analogs of this invention can be used for as an active ingredient in pharmaceutical compositions for both medical applications and animal husbandry or veterinary applications. Typically, the HBGF analog or pharmaceutical composition is used in humans, but may also be used in other mammals. The term “patient” is intended to denote a mammalian individual, and is so used throughout the specification and in the claims. The primary applications of this invention involve human patients, but this invention may be applied to laboratory, farm, zoo, wildlife, pet, sport or other animals.

The HBGF analogs of this invention may be in the form of any pharmaceutically acceptable salt. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the HBGF analog of the present invention is basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts of the HBGF analogs of this invention are prepared in a suitable solvent for the HBGF analog and an excess of an acid, such as hydrochloric, hydrobromic, sulfuric, phosphoric, acetic, trifluoroacetic, citric, tartaric, maleic, succinic or methanesulfonic acid. The acetate salt form is especially useful. Where the HBGF analogs of this invention include an acidic moiety, suitable pharmaceutically acceptable salts may include alkali metal salts, such as sodium or potassium salts, or alkaline earth metal salts, such as calcium or magnesium salts.

The invention provides a pharmaceutical composition that includes a HBGF analog of this invention and a pharmaceutically acceptable carrier. The carrier may be a liquid formulation, and in one embodiment a buffered, isotonic, aqueous solution. Pharmaceutically acceptable carriers also include excipients, such as diluents, carriers and the like, and additives, such as stabilizing agents, preservatives, solubilizing agents, buffers and the like, as hereafter described.

Thus the HBGF analog compositions of this invention may be formulated or compounded into pharmaceutical compositions that include at least one HBGF analog of this invention together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, PEG, PEO, mannitol, sodium chloride or sodium citrate, as well as any number of simple sugars, including sucrose, dextrose, lactose and the like, and combinations of the foregoing. For injection or other liquid administration formulations, water containing at least one or more buffering constituents is preferred, and stabilizing agents, preservatives and solubilizing agents may also be employed. For solid administration formulations, any of a variety of thickening, filler, bulking and carrier additives may be employed, such as starches, sugars, fatty acids and the like. For topical administration formulations, any of a variety of creams, ointments, gels, lotions and the like may be employed. For most pharmaceutical formulations, non-active ingredients will constitute the greater part, by weight or volume, of the preparation. For pharmaceutical formulations, it is also contemplated that any of a variety of measured-release, slow-release or time-release formulations and additives may be employed, so that the dosage may be formulated so as to effect delivery of a HBGF analog of this invention over a period of time.

In practical use, the HBGF analogs of the invention can be combined as the active ingredient in an admixture with a pharmaceutical carrier according to conventional pharmaceutical compounding techniques. The carrier may take a wide variety of forms depending on the form of preparation desired for administration, for example, oral, parenteral (including intravenous), urethral, vaginal, nasal, buccal, sublingual, or the like. In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that it may be administered by syringe. The form must be stable under the conditions of manufacture and storage and must be preserved against the contaminating action of microorganisms such as bacteria and fungi. The carrier can be a solvent or dispersion medium containing, for example, water, ethanol, a polyol, for example glycerol, propylene glycol or liquid polyethylene glycol, suitable mixtures thereof, and vegetable oils.

If the HBGF analog pharmaceutical composition is administered by injection, the injection may be intravenous, subcutaneous, intramuscular, intraperitoneal or other means known in the art. The HBGF analogs of this invention may alternatively be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the HBGF analogs of invention are introduced across an epidermal layer of cells may be employed. Administration means may thus include administration through mucous membranes, buccal administration, oral administration, dermal administration, inhalation administration, nasal administration, urethral administration, vaginal administration, and the like.

In general, the actual quantity of HBGF analog of this invention administered to a patient will vary between fairly wide ranges depending upon the mode of administration, the formulation used, and the response desired. The dosage for treatment is administration, by any of the foregoing means or any other means known in the art, of an amount sufficient to bring about the desired therapeutic effect.

### Heparin-Binding Growth Factors

The fibroblast growth factors, FGFs, constitute a family of related proteins controlling normal growth and differentiation of mesenchymal, epithelial, and neuroectodermal cell types. Homologs have been found in a wide variety of species. FGFs show a very high affinity to heparin and are therefore also referred to as heparin-binding growth factors (HBGFs). As used herein, the term HBGFs includes all FGFs.

Two main types of FGF are known. The first type of FGF was isolated initially from brain tissue. It was identified by its proliferation-enhancing activities for murine fibroblasts, such as 3T3 cells. Due to its basic pI the factor was named basic FGF (bFGF, or HBGF-2, heparin-binding growth factor-2) and is now generally referred to as FGF-2. This is the prototype of the FGF family.

Another type of FGF, also initially isolated from brain tissues, is acidic FGF (aFGF, also known as HBGF-1, heparin-binding growth factor-1 or HBGF-α, heparin-binding growth factor-α), now generally referred to as FGF-1. It was identified by its proliferation-enhancing activity for myoblasts.

Other fibroblast growth factors belonging to the same family include FGF-3 (or HBGF-3, heparin-binding growth factor-3, originally called int-2; see Fekete, Trends in Neurosci. 23:332 (2000)), FGF-4 (HBGF-4, heparin-binding growth factor-4, initially recognized as the product of the oncogene hst; see Sakamoto et al., Proc. Natl. Acad. Sci. USA 91:12368-72), and FGF-5 (originally called HBGF-5, see Bates et al. Biosynthesis of human fibroblast growth factor 5. Mol. Cell. Biol. 11:1840-1845 (1991); Burgess and Maciag, The heparin-binding (fibroblast) growth factor family of proteins. Ann. Rev. Biochem. 58: 575-606 (1989); and Zhan et al. The human FGF-5 oncogene encodes a novel protein related to fibroblast growth factors. Mol. Cell. Biol. 8:3487-3495 (1988)).

FGF-6 is also known as HBGF-6, and sometimes called hst-2 or oncogene hst-1 related growth factor, see Iida et al. Human hst-2 (FGF-6) oncogene: cDNA cloning and characterization. Oncogene 7:303-9 (1992); and Marics et al. Characterization of the HST-related FGF-6 gene, a new member of the fibroblast growth factor gene family. Oncogene 4:335-40 (1989).

FGF-7 or K-FGF is also known as KGF or keratinocyte growth factor (See Aaronson et al. Keratinocyte growth factor. A fibroblast growth factor family member with unusual target cell specificity. Annals NY Acad. Sci. 638:62-77 (1991); Finch et al. Human KGF is FGF-related with properties of a paracrine effector of epithelial cell growth. Science 245:752-5 (1989); Marchese et al. Human keratinocyte growth factor activity on proliferation and differentiation of human keratinocytes: differentiation response distinguishes KGF from EGF family. J. Cellular Physiol. 144: 326-32 (1990)).

FGF-8 was found to be identical to androgen-induced growth factor, AIGF and has been well studied (See Blunt et al. Overlapping expression and redundant activation of mesenchymal fibroblast growth factor (FGF) receptors by alternatively spliced FGF-8 ligands. J. Biol. Chem. 272:3733-8 (1997); Dubrulle et al. FGF signaling controls somite boundary position and regulates segmentation clock control of spatiotemporal Hox gene activation. Cell 106:219-232 (2001); Gemel et al. Structure and sequence of human FGF8. Genomics 35:253-257 (1996); Tanaka et al. A novel isoform of human fibroblast growth factor 8 is induced by androgens and associated with progression of esophageal carcinoma. Dig. Dis. Sci. 46:1016-21 (2001)).

FGF-9 was originally called glia activating factor, or HBGF-9. See Miyamoto et al. Molecular cloning of a novel cytokine cDNA encoding the ninth member of the fibroblast growth factor family, which has a unique secretion pattern. Mol. Cell. Biol. 13:4251-9 (1993); and Naruo et al. Novel secretory heparin-binding factors from human glioma cells (glia-activating factors) involved in glial cell growth. J. Biol. Chem. 268: 2857-64 (1993).

FGF-10 is also called KGF-2, keratinocyte growth factor-2 (see Kok et al. Cloning and characterization of a cDNA encoding a novel fibroblast growth factor preferentially expressed in human heart. Biochem. Biophys. Res. Comm. 255:717-721, (1999)).

Several FGF-related factors have been described as fibroblast growth factor homologous factors (FHFs) and are also referred to as FGF-11 (FHF-3), FGF-12 (FHF-1), FGF-13 (FHF-2, see Greene et al. Identification and characterization of a novel member of the fibroblast growth factor family. Eur. J. Neurosci. 10:1911-1925 (1998)), and FGF-14 (FHF-4).

FGF-15 is expressed in the developing nervous system and was identified as a gene regulated by transcription factor E2A-Pbx1. McWhirter et al. A novel fibroblast growth factor gene expressed in the developing nervous system is a downstream target of the chimeric homeodomain oncoprotein E2A-Pbx1. Development 124:3221-3232 (1997).

FGF-16 was isolated as a cDNA clone from rat heart by homology-based polymerase chain reaction expressing an FGF of 207 amino acids. FGF-16 is 73% identical to FGF-9. Miyake et al. Structure and expression of a novel member, FGF-16, of the fibroblast growth factor family. Biochem. Biophys. Res. Commun. 243:148-152 (1998).

The cDNA encoding FGF-17 was isolated from rat embryos and encodes a protein of 216 amino acids. When expressed in 3T3 fibroblasts, mouse FGF-17 is transforming. During embryogenesis, FGF-17 is expressed at specific sites in forebrain, the midbrain-hindbrain junction, the developing skeleton and in developing arteries. See Hoshikawa et al. Structure and expression of a novel fibroblast growth factor, FGF-17, preferentially expressed in the embryonic brain. Biochem. Biophys. Res. Commun. 244:187-191 (1998); and Xu et al. Genomic structure, mapping, activity and expression of fibroblast growth factor 17. Mechanisms of Development 83:165-178 (1999).

The cDNA encoding FGF-18 was isolated from rat embryos encoding a protein of 207 amino acids. FGF-18 is a glycosylated protein and is most similar to FGF-8 and FGF-17. Injection of recombinant murine FGF-18 has been shown to induce proliferation in tissues of both epithelial and mesenchymal origin, particularly in liver and small intestine. Recombinant rat FGF-18 induces neurite outgrowth in PC12 cells. Recombinant murine FGF-18 protein stimulates proliferation in NIH 3T3 fibroblasts in vitro in a heparan sulfate-dependent manner. For general information see Hu et al. FGF-18, a novel member of the fibroblast growth factor family, stimulates hepatic and intestinal proliferation. Mol. Cell. Biol. 18:6063-6074 (1998); and Ohbayashi et al. Structure and expression of the mRNA encoding a novel fibroblast growth factor, FGF-18. J. Biol. Chem. 273:18161-18164 (1998).

FGF-19 is related distantly to other members of the FGF family. FGF-19 mRNA is expressed in several tissues including fetal cartilage, skin, and retina, as well as adult gall bladder. It is overexpressed in a colon adenocarcinoma cell line. FGF-19 is a high affinity, heparin-dependent ligand for the FGF-4 receptor. See Xie et al. FGF-19, a novel fibroblast growth factor with unique specificity for FGFR4 Cytokine 11:729-735 (1999).

FGF-20 is expressed in normal brain, particularly the cerebellum, and in some cancer cell lines. FGF-20 mRNA is expressed preferentially in the substantia nigra pars compacta. Recombinant FGF-20 protein induces DNA synthesis in a variety of cell types and is recognized by multiple FGF receptors. FGF-20 functions like an oncogene, causing a transformed phenotype when expressed in the 3T3 fibroblast cell line. These transformed cells are tumorigenic in nude mice. See Jeffers et al. Identification of a novel human fibroblast growth factor and characterization of its role in oncogenesis. Cancer Res. 61:3131-8 (2001); and Ohmachi et al. FGF-20, a novel neurotrophic factor, preferentially expressed in the substantia nigra pars compacta of rat brain. Biochem. Biophys. Res. Commun. 277:355-60 (2000).

FGF-21 was isolated from mouse embryos. FGF-21mRNA is most abundant in the liver with lower levels in the thymus. FGF-21 is most similar to human FGF-19. See Nishimura et al. Identification of a novel FGF, FGF-21, preferentially expressed in the liver. Biochim. Biophys. Acta 1492:203-6 (2000).

The cDNA encoding FGF-22 (170 amino acids) was isolated from human placenta. FGF-22 is most similar to FGF-10 and FGF-7. Murine FGF-22 mRNA is expressed preferentially in the skin. FGF-22 mRNA in the skin is found preferentially in the inner root sheath of the hair follicle. See Nakatake et al. Identification of a novel fibroblast growth factor, FGF-22, preferentially expressed in the inner root sheath of the hair follicle. Biochim. Biophys. Acta 1517:460-3 (2001).

FGF-23 is most similar to FGF-21 and FGF-19. The human FGF-23 gene maps to chromosome 12p13 linked to human FGF-6 gene. FGF-23 mRNA is expressed mainly in the brain (preferentially in the ventrolateral thalamic nucleus) and thymus at low levels. Missense mutations in the FGF-23 gene have been found in patients with autosomal dominant hypophosphataemic rickets. Overproduction of FGF23 causes tumor-induced osteomalacia, a paraneoplastic disease characterized by hypophosphatemia caused by renal phosphate wasting. See Yamashita et al. Identification of a novel fibroblast growth factor, FGF-23, preferentially expressed in the ventrolateral thalamic nucleus of the brain. Biochem. Biophys. Res. Commun. 277:494-8 (2000); and Shimada et al. Cloning and characterization of FGF23 as a causative factor of tumor-induced osteomalacia. Proc. Natl. Acad. Sci. (USA) 98:6500-5 (2001).

HBBM (Heparin-binding brain mitogen) was isolated initially as a heparin binding protein from brain tissues of several species and is identical to heparin-binding neurite promoting factor. See Huber et al. Amino-terminal sequences of a novel heparin-binding protein with mitogenic activity for endothelial cells from human bovine, rat, and chick brain: high interspecies homology. Neurochem. Res. 15:435-439 (1990).

HB-GAF (heparin-binding growth associated factor) is a neurotrophic and mitogenic factor identical to HBNF (heparin-binding neurite-promoting factor). See Kuo et al. Characterization of heparin-binding growth-associated factor receptor in NIH 3T3 cells. Biochem. Biophys. Res. Commun. 182:188-194 (1992).

HB-EGF (heparin-binding EGF-like factor) is found in conditioned media of cell line U937 and is also synthesized by macrophages and human vascular smooth muscle cells. HB-EGF is a monomeric heparin-binding O-glycosylated protein of 86 amino acids and is processed from a precursor of 208 amino acids. Several truncated forms of HB-EGF have been described. HB-EGF is a potent mitogen for NIH 3T3 cells, keratinocytes and smooth muscle cells, but not for endothelial cells. The mitogenic activity on smooth muscle cells is much stronger than for EGF and appears to involve interactions with cell surface heparan sulfate proteoglycans. HB-EGF is a major growth factor component of wound fluid and may play an important role in wound healing. See Abraham et al. Heparin-binding EGF-like growth factor: characterization of rat and mouse cDNA clones, protein domain conservation across species, and transcript expression in tissues. Biochem. Biophys. Res. Commun. 190:125-133 (1993); Higashiyama et al. A heparin-binding growth factor secreted by macrophage like cells that is related to EGF. Science 251:936-9 (1991); and Marikovsky et al. Appearance of heparin-binding EGF-like growth factor in wound fluid as a response to injury. Proc. Natl. Acad. Sci. (USA) 90:3889-93.

HB-GAM (heparin-binding growth associated molecule) also referred to as HBNF (heparin-binding neurite promoting factor) is a protein of 15.3 kDa isolated as a heparin binding protein from brain tissues of several species. HB-GAM promotes growth of SW-13 cells in soft agar. Courty et al. Mitogenic properties of a new endothelial cell growth factor related to pleiotrophin. Biochem. Biophys. Res. Commun. 180:145-151 (1991); and Hampton et al. Structural and functional characterization of full-length heparin-binding growth associated molecule. Mol. Biol. Cell. 3:85-93 (1992).

TGF-beta (TGF-β) exists in at least five isoforms, known TGF-β1, TGF-β2, TGF-β3, TGF-β4 and TGF-β5, that are not related to TGF-α. Their amino acid sequences display homologies on the order of 70-80 percent. TGF-β1 is the prevalent form and is found almost ubiquitously while the other isoforms are expressed in a more limited spectrum of cells and tissues.

TGF-beta is the prototype of a family of proteins known as the TGF-beta superfamily. This family includes inhibins, Activin A, MIS (Mullerian activating substance) and BMPs (Bone morphogenic proteins). Burt, Evolutionary grouping of the transforming growth factor-beta superfamily. Biochem. Biophys. Res. Commun. 184:590-5 (1992).

### EXAMPLES

### Example 1

A synthetic branched peptide construct of the general formula: NH₂-LYVDFSDVGWNDWC(bMB-CX₁X₁X₁X₁RKRLDRIAR-amide)-LYVDFSDVGWNDW-amide, where bMB is 1,4-bis-maleimidobutane and each X₁ is 6-aminohexanoic acid, is synthesized using two peptide precursors. The first peptide precursor is CX₁X₁X₁RKRLDRIAR-amide (SEQ ID NO:56), again where each X₁ is 6-aminohexanoic acid, while the second peptide precursor is LYVDFSDVGWNDWCLYVDFSDVGWNDW-amide (SEQ ID NO:57). Both peptide precursors are synthesized by conventional solid phase methods and purified by reverse-phase HPLC. In the first peptide precursor, CX₁X₁X₁RKRLDRIAR-amide (SEQ ID NO:56), the cysteine residue serves to link the precursor to 1,4-bis-maleimidobutane, the 6-aminohexanoic acid X₁X₁X₁ tripeptide serves as both a spacer and provided a hydrophobic region, and the sequence RKRLDRIAR (SEQ ID NO:58) provides a heparin-binding motif derived from a modification of the sequence at residues 270-279 of the Jun/AP-1 DNA binding domain (Busch et al. Trans-Repressor Activity of Nuclear Glycosaminoglycans on Fos and Jun/AP-1 Oncoprotein-mediated Transcription. J. Cell Biol. 116:31-42, 1992).

In the second peptide precursor LYVDFSDVGWNDWCLYVDFSDVGWNDW-amide (SEQ ID NO:57), the sequence LYVDFSDVGWNDW (SEQ ID NO:27) is derived from amino acids 19-31 in the human BMP-2 sequence and the cysteine residue served to link the precursor to 1,4-bis-maleimidobutane.

Synthetic Scheme 1 shows the general approach for synthesis. The first precursor CX₁X₁X₁RKRLDRIAR-amide (SEQ ID NO:56) is reacted in a slightly acidic conditions with a large molar excess of A, 1,4-bis-maleimidobutane, a homobifunctional, sulfhydryl reactive agent with a four carbon spacer, in a 50% acetonitrile/water adjusted to 5.8 pH by use of 0.1M sodium phosphate dibasic buffer. In the depiction of the resulting reaction product B, “S¹” depicts the sulfur atom “S” of the side chain of the cysteine residue, shown as “C”. The reaction product B is isolated by preparative HPLC and the isolated reaction product B is then reacted with LYVDFSDVGWNDWCLYVDFSDVGWNDW-amide (SEQ ID NO:57), again in a 50% acetonitrile/water adjusted to 8.0 pH by use of 0.1 M sodium phosphate dibasic. Here too in the depiction of the resulting final product C, “S²” depicts the sulfur atom “S” of the side chain of the cysteine residue, shown as “C”, forming a part of SEQ ID NO:57. The final product C is purified by HPLC, using a 250×10.0 mm C₁₈ column at a flow rate of 3 mL/min in a solvent gradient of 0 to 60% of 0.1% trifluoroacetate in acetonitrile where the initial solvent is 0.1% trifluoroacetate and water. The resulting final product C is a branched peptide construct wherein the two peptide subunits are conjugated via thioether linkages. Mass spectroscopy is used to confirm the sequence of the final construct.

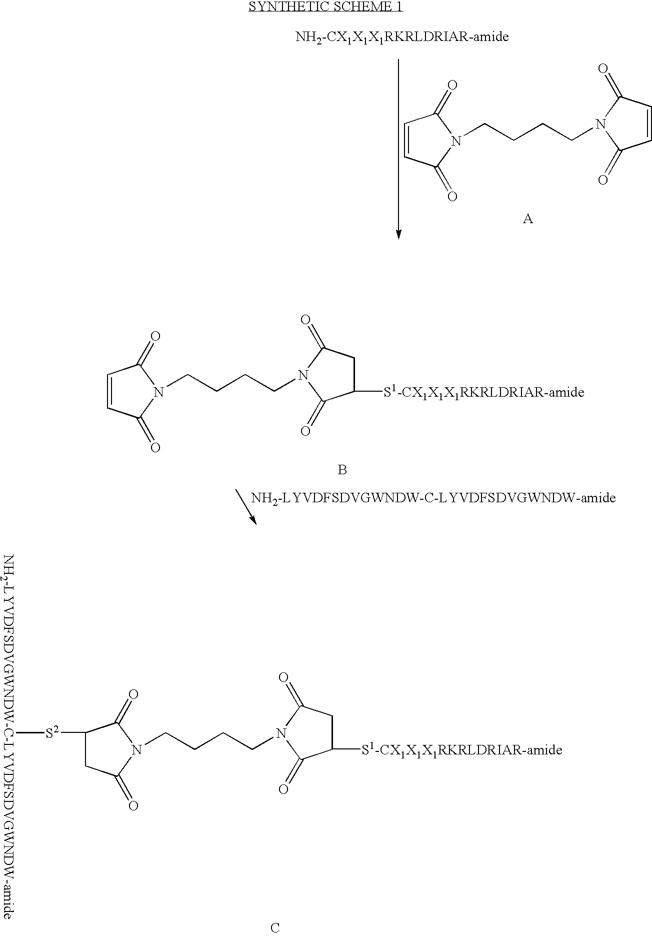

It may thus be seen that the structure of the molecule may alternatively be depicted as shown below, where amino acid residues are shown by bolded single letter designations, letter designations that are not bolded refer to atoms, and X₁ is in each instance 6-aminohexanoic acid:

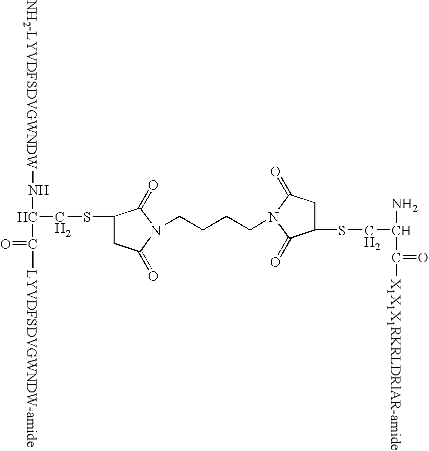

### Example 2

The synthetic branched peptide construct of Example 1 is tested in cell growth studies, to determine the ability of the synthetic branched peptide construct to stimulate cell growth. Cell growth is monitored using a commercially available kit (Promega Corporation, Madison, Wis.) based on a tetrazolium compound (3-(4,5-dimethylthiazol-2-yl)-5-(3-carboxymethoxyphenyl)-2-(4-sulfophenyl)-2H-tetrazolium, inner salt (MTS). Aliquots of 10³ cells are seeded into wells of 96-well plates and allowed to attach. The medium is then replaced with a low serum medium containing between 0 and 10 μg/mL of the synthetic branched peptide construct of Example 1. After 3 days in culture, the relative cell number is monitored using MTS following the directions of the manufacturer. Statistical significance is determined using ANOVA followed by post-hoc multiple comparisons versus control group (Dunnett's Method).

MC3T3E1 osteoblastic cells, C3H10T1/2 mouse pluripotent stem cells, C2C12 murine myoblasts, and cells from a human fetal osteoblast cell line (hFOB) were obtained from the American Type Culture Collection (Manassas, Va.) and are maintained in DMEM:F12 medium containing newborn calf serum and antibiotics. MC3T3E1, C3H10T1/2, C2C12 and hFOB cells are tested for growth induction by the synthetic branched peptide construct of Example 1.

### Example 3

In an alternative method of synthesis, a first peptide precursor chain NH₂—C(Npys)X₁X₁X₁RKRLDRIAR-amide (SEQ ID NO:59) is synthesized by conventional peptide synthesis methods, where each X₁ is 6-aminohexanoic acid and C(Npys) is S-(3-nitro-2-pyridinesulfenyl)-cysteine. The second peptide precursor chain AISMLYLDENEKVVLCAISMLYLDENEKVVL-amide (SEQ ID NO:60), is synthesized as in Example 1. The sequence AISMLYLDENEKVVL (SEQ ID NO:28) is derived from amino acids 87-100 in the human BMP-2 sequence. As shown in Synthetic Scheme 2, the two chains are reacted, resulting in a disulfide bond between the two sulfur atoms (S¹ and S²) of the cysteine residues of SEQ ID NO. 59 and SEQ ID NO:60, respectively.

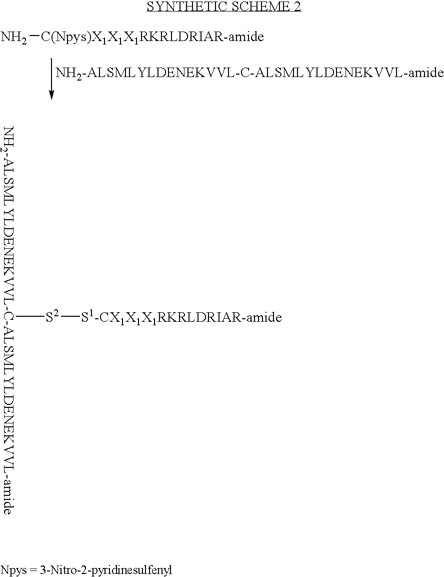

### Example 4

A synthetic branched peptide construct of the general formula: NH₂-LYVDFSDVGWNDWC(bMB-CX₁X₁X₁RKRLDRIAR-amide)-AISMLYLDENEKVVL-amide, where bMB is 1,4-bis-maleimidobutane and each X₁ is 6-aminohexanoic acid, was synthesized using two peptide precursors. The first peptide precursor was CX₁X₁X₁RKRLDRIAR-amide (SEQ ID NO:58), again where each X₁ is 6-aminohexanoic acid, while the second peptide precursor was LYVDFSDVGWNDWCAISMLYLDENEKVVL-amide (SEQ ID NO:59). Both peptide precursors were synthesized by conventional solid phase methods and purified by reverse-phase HPLC. In the first peptide precursor, CX₁X₁X₁RKRLDRIAR-amide (SEQ ID NO:58), the cysteine residue served to link the precursor to 1,4-bis-maleimidobutane, the 6-aminohexanoic acid X₁X₁X₁ tripeptide served as both a spacer and provided a hydrophobic region, and the sequence RKRLDRIAR (SEQ ID NO:60) provided a heparin-binding motif derived from a modification of the sequence at residues 270-279 of the Jun/AP-1 DNA binding domain (Busch et al. Trans-Repressor Activity of Nuclear Glycosaminoglycans on Fos and Jun/AP-1 Oncoprotein-mediated Transcription. J. Cell Biol. 116:31-42, 1992).

In the second peptide precursor LYVDFSDVGWNDWCAISMLYLDENEKVVL-amide (SEQ ID NO:59), the sequence LYVDFSDVGWNDW (SEQ ID NO:27) was derived from amino acids 19-31 in the human BMP-2 sequence, the cysteine residue served to link the precursor to 1,4-bis-maleimidobutane, and the sequence AISMLYLDENEKVVL (SEQ ID NO:28) was derived from amino acids 87-100 in the human BMP-2 sequence.

Synthetic Scheme 3 shows the general approach for synthesis. The first precursor CX₁X₁X₁RKRLDRIAR-amide (SEQ ID NO:58) was reacted in a slightly acidic conditions with a large molar excess of A, 1,4-bis-maleimidobutane, a homobifunctional, sulfhydryl reactive agent with a four carbon spacer, in a 50% acetonitrile/water adjusted to 5.8 pH by use of 0.1M sodium phosphate dibasic buffer. In the depiction of the resulting reaction product B, “S¹” depicts the sulfur atom “S” of the side chain of the cysteine residue, shown as “C”. The reaction product B was isolated by preparative HPLC and the isolated reaction product B was then reacted with LYVDFSDVGWNDWCALSMLYLDENEKVVL-amide (SEQ ID NO:59), again in a 50% acetonitrile/water adjusted to 8.0 pH by use of 0.1M sodium phosphate dibasic. Here too in the depiction of the resulting final product C, “S²” depicts the sulfur atom “S” of the side chain of the cysteine residue, shown as “C”, forming a part of SEQ ID NO:59. The final product C was purified by HPLC, using a 250×10.0 mm C₁₈ column at a flow rate of 3 mL/min in a solvent gradient of 0 to 60% of 0.1% trifluoroacetate in acetonitrile where the initial solvent was 0.1% trifluoroacetate and water. The resulting final product C was a branched peptide construct wherein the two peptide subunits were conjugated via thioether linkages. Mass spectroscopy was used to confirm the sequence of the final construct.

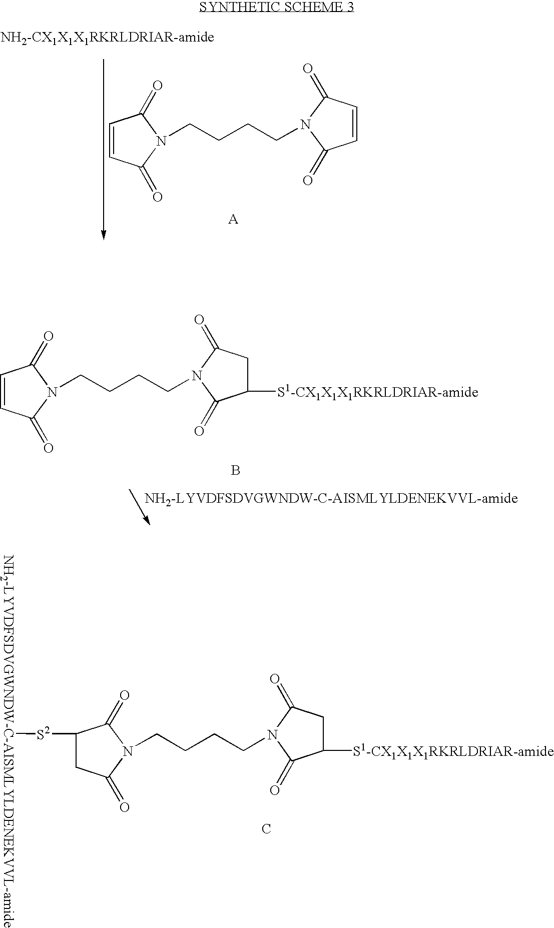

It may thus be seen that the structure of the molecule may alternatively be depicted as shown in FIG. 2, where amino acid residues are shown by bolded single letter designations, letter designations that are not bolded refer to atoms, and X₁ is in each instance 6-aminohexanoic acid.

### Example 5

The synthetic branched peptide construct of Example 4 was tested in cell growth studies, to determine the ability of the synthetic branched peptide construct to stimulate cell growth. Cell growth was monitored using a commercially available kit (Promega Corporation, Madison, Wis.) based on a tetrazolium compound (3-(4,5-dimethylthiazol-2-yl)-5-(3-carboxymethoxyphenyl)-2-(4-sulfophenyl)-2H-tetrazolium, inner salt (MTS). Aliquots of 10³ cells were seeded into wells of 96-well plates and allowed to attach. The medium was then replaced with a low serum medium containing between 0 and 10 μg/mL of the synthetic branched peptide construct of Example 4. After 3 days in culture, the relative cell number was monitored using MTS following the directions of the manufacturer. Statistical significance was determined using ANOVA followed by post-hoc multiple comparisons versus control group (Dunnett's Method).

MC3T3E1 osteoblastic cells, C3H10T1/2 mouse pluripotent stem cells, C2C12 murine myoblasts, and cells from a human fetal osteoblast cell line (hFOB) were obtained from the American Type Culture Collection (Manassas, Va.) and were maintained in DMEM:F12 medium containing newborn calf serum and antibiotics. MC3T3E1, C3H10T1/2, and hFOB cells were positive for growth induction by synthetic branched peptide construct of Example 2 as shown in Table 3 below. The effective concentration was between 1 and 10 μg/mL. C2C12 was negative for growth using the synthetic branched peptide construct of Example 4.

TABLE 3

| | | MEAN | | | % OF |
|---|---|---|---|---|---|
| | Compound | ABSOR- | | | CONTROL |
| Cell line | μg/mL | BANCE | S.D. | P value | VALUE |
| hFOB | 0 | 0.48 | 0.08 | | 100 |
| | 0.5 | 0.58 | 0.07 | ns | 121 |
| | 1 | 0.62 | 0.04 | <0.05 | 131 |
| | 2 | 0.64 | 0.03 | <0.05 | 135 |
| | 5 | 0.60 | 0.03 | <0.05 | 127 |
| | 10 | 0.62 | 0.04 | <0.05 | 130 |
| MC3T3E1 | 0 | 0.72 | 0.088 | | 100 |
| | 0.5 | 0.74 | 0.100 | ns | 103 |
| | 1 | 0.71 | 0.097 | ns | 99 |
| | 2 | 0.99 | 0.124 | <0.05 | 138 |
| | 5 | 0.96 | 0.129 | <0.05 | 133 |
| | 10 | 0.96 | 0.234 | <0.05 | 134 |
| C3H10T1/2 | 0 | 0.28 | 0.04 | | 100 |
| | 0.5 | 0.41 | 0.06 | <0.05 | 147 |
| | 1 | 0.42 | 0.05 | <0.05 | 152 |
| | 2 | 0.44 | 0.03 | <0.05 | 158 |
| | 5 | 0.43 | 0.03 | <0.05 | 156 |
| | 10 | 0.40 | 0.03 | ns | 144 |
| C2C12 | 0 | 0.30 | 0.02 | | 100 |
| | 0.5 | 0.30 | 0.01 | ns | 99 |
| | 1 | 0.30 | 0.01 | ns | 101 |
| | 2 | 0.30 | 0.01 | ns | 99 |
| | 5 | 0.30 | 0.01 | ns | 99 |
| | 10 | 0.27 | 0.01 | ns | 90 |

### Example 6

In an alternative method of synthesis, a first peptide precursor chain NH₂—C(Npys)X₁X₁X₁RKRLDRIAR-amide (SEQ ID NO:61) is synthesized by conventional peptide synthesis methods, where each X₁ is 6-aminohexanoic acid and C(Npys) is S-(3-nitro-2-pyridinesulfenyl)-cysteine. The second peptide precursor chain LYVDFSDVGWNDWCAISMLYLDENEKVVL-amide (SEQ ID NO:59), is synthesized as in Example 4. As shown in Synthetic Scheme 4, the two chains are reacted, resulting in a disulfide bond between the two sulfur atoms (S¹ and S²) of the cysteine residues of SEQ ID NO. 61 and SEQ ID NO:59, respectively.

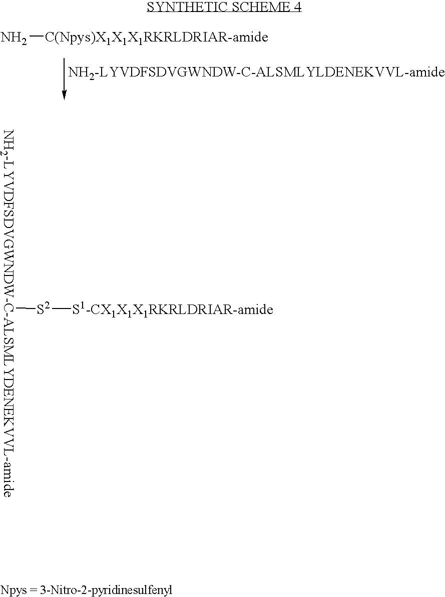

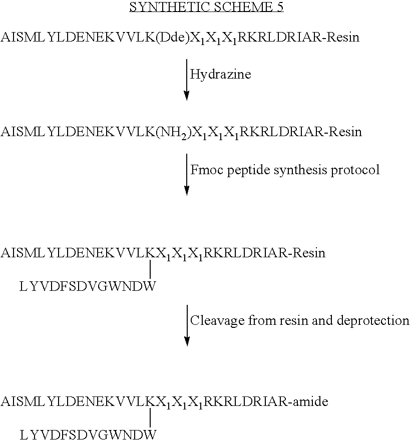

The preceding examples can be repeated with similar success by substituting the generically or specifically described peptide sequences, reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

The present invention has been described in terms of preferred embodiments, however, it will be appreciated that various modifications and improvements may be made to the described embodiments without departing from the scope of the invention.

What is claimed is:

1. A heparin-binding growth factor analog of formula I: 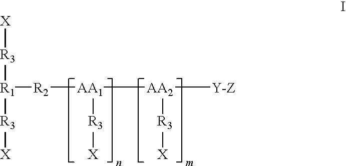
wherein:
each X a peptide chain that (i) has a minimum of three amino acid residues, (ii) has a maximum of about fifty amino acid residues, and (iii) binds a heparin-binding growth factor receptor (HBGFR);
R₁ is a trifunctional amino acid residue covalently bonded to one X through the N terminus amine of R₁ and the remaining X through the C terminus carboxyl of R₁;
R₂ is a linker comprising a chain from 3 to about 20 atoms covalently bonded to the side chain of R₁ and Y when n=0, or to the side chain of R₁ and to the N terminus amine of AA₁ when n=1;
Each R₃ is independently from 0 to about 3 amino acid residues which are the same or different;
Y is a linker comprising a chain from 0 to about 50 atoms covalently bonded to R₂ and Z when n=0, or to AA₁ and Z when n=1 and m=0, or to AA₂ and Z when n=1 and m=1;
Z is a non-signaling peptide chain that includes a heparin binding domain, comprising an amino acid sequence that comprises (i) a minimum of one heparin binding motif, (ii) a maximum of about ten heparin binding motifs, and (iii) a maximum of about thirty amino acids;
AA₁ and AA₂ are each independently a trifunctional amino acid residue, wherein X is covalently bonded through the side chain of AA₁ and AA₂; and
n is 0 or 1, and when n is 1, m is 0 or 1, and when n is 0, m is 0.

2. The heparin-binding growth factor analog of claim 1 wherein X and Z are synthetic peptide chains.

3. The heparin-binding growth factor analog of claim 1 wherein Y further comprises a linker that (i) is hydrophobic, (ii) comprises a chain of a minimum of about 9 and a maximum of about 50 atoms, and (iii) is not found in the natural ligand of the heparin-binding growth factor receptor (HBGFR) which X binds.

4. The heparin-binding growth factor analog of claim 1 wherein the heparin-binding growth factor analog has an avidity for heparin such that the synthetic heparin-binding growth factor analog binds heparin in 0.15 M NaCl, but is eluted by 1 M NaCl.

5. The heparin-binding growth factor analog of claim 1 wherein the construct is of formula II: 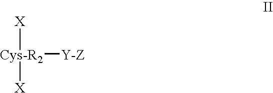
wherein:
Cys is cysteine; and
R₂ is a linker consisting of a sulfhydryl reactive homo-bifunctional cross-linker and a second Cys or comprising a hetero-bifunctional cross-linker.

6. The heparin-binding growth factor analog of claim 5 wherein the construct is of formula III: 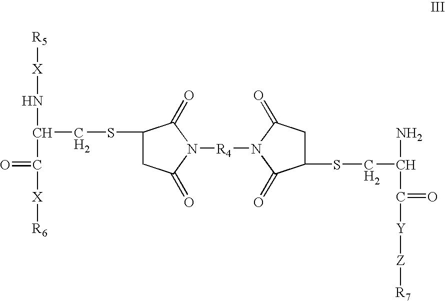
wherein:
R₄ is a linker comprising a chain of between 1 and about 10 backbone atoms selected from carbon, oxygen, sulfur and nitrogen or mixtures thereof;
R₅ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative;
R₆ is OH, NH₂, or NH—R₅; and
R₇ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative.

7. The heparin-binding growth factor analog of claim 6 wherein the construct is of formula IV: 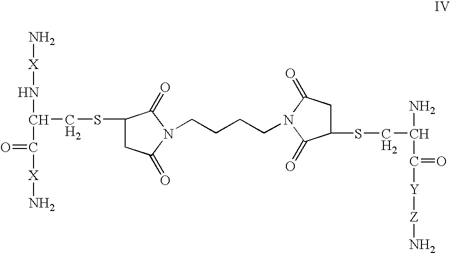
wherein:
X is any of SEQ ID NOS:6-55;
Y is between two and about five amino acid residues selected from the group consisting of 6-aminohexanoic acid, 7-aminoheptanoic acid, 9-aminononanoic acid and mixtures thereof; and
Z is any of SEQ ID NOS:2-5 or SEQ ID NO:58.

8. The heparin-binding growth factor analog of claim 1 wherein the covalent bonds between the side chain of R₁ and R₂ and between R₂ and Z when n=0, or between AA₁ and Z when n=1 and m=0, or between AA₂ and Z when n=1 and m=1, comprise an amide, disulfide, thioether, Schiff base, reduced Schiff base, imide, secondary amine, carbonyl, urea, hydrazone or oxime bond.

9. The heparin-binding growth factor analog of claim 1 wherein the side chains of AA₁ and AA₂ comprise reactive carboxyl groups.

10. The heparin-binding growth factor analog of claim 1 wherein the side chain of R₁ comprises a reactive sulfhydryl group.

11. The heparin-binding growth factor analog of claim 10 wherein R₁ is an L- or D-3-mercapto amino acid.

12. The heparin-binding growth factor analog of claim 11 wherein the L- or D-3-mercapto amino acid is L- or D-cysteine, L- or D-penicillamine, 3-mercapto phenylalanine, or a derivative of any of the foregoing.

13. The heparin-binding growth factor analog of claim 1 wherein X is any of SEQ ID NO:5 to SEQ ID NO:55 and Z is RKRLDRIAR (SEQ ID NO:58), RKRKLERIAR (SEQ ID NO:2) RKRKLGRIAR (SEQ ID NO:3) or RKRKLWRARA (SEQ ID NO:4).

14. The heparin-binding growth factor analog of claim 1 wherein one or more of R₃ is between one and about three amino acid residues selected from the group consisting of glycine, 6-aminohexanoic acid, 7-aminoheptanoic acid, 9-aminononanoic acid and mixtures thereof.

15. The heparin-binding growth factor analog of claim 1 wherein X comprises an amino acid sequence found in any of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, glial derived neurotrophic factor (GDNF), GRO2, hepatoma-derived growth factor (HDGF), and granulocyte-macrophage colony stimulating factor (GMCSF), a homolog of any amino acid sequence found in the foregoing, a reverse sequence of any amino acid sequence found in the foregoing, or a homolog of a reverse sequence of any amino acid residue found in the foregoing.

16. The heparin-binding growth factor analog of claim 1 wherein Y comprises between one and about thirty-three ethylene glycol units.

17. The heparin-binding growth factor analog of claim 1 wherein Y comprises a branched or unbranched, saturated or unsaturated alkyl chain of between one and about twenty carbon atoms.

18. The heparin-binding growth factor analog of claim 1 wherein Y comprises [NH₂—(CH₂)ₚCO]_{q} wherein p is from 1 to about 10 and q is from 1 to about 20.

19. The heparin-binding growth factor analog of claim 1 wherein Y comprises a peptide sequence comprising from one to about 16 Gly residues.

20. The heparin-binding growth factor analog of claim 1 wherein each heparin binding motif of Z is BxBB or BBBxxB, wherein each B is independently lysine, arginine, ornithine, or histidine, and each x is a independently a naturally occurring amino acid.

21. The heparin-binding growth factor analog of claim 20 wherein Z comprises at least two heparin-binding motifs.

22. A pharmaceutical composition comprising the heparin-binding growth factor analog of claim 1 or a pharmaceutically acceptable salt thereof and a pharmaceutical carrier.

23. A heparin-binding growth factor analog of formula V: 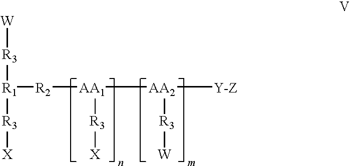
wherein:
each X and each W is a peptide chain differing by at least one amino acid residue that (i) has a minimum of three amino acid residues, (ii) has a maximum of about fifty amino acid residues, and (iii) binds a heparin-binding growth factor receptor (HBGFR);
R₁ is a trifunctional amino acid residue covalently bonded to one X through the N terminus amine of R₁ and the remaining X through the C terminus carboxyl of R₁;
R₂ is a linker comprising a chain from 3 to about 20 atoms covalently bonded to the side chain of R₁ and Y when n=0, or to the side chain of R₁ and to the N terminus amine of AA₁ when n=1;
Each R₃ is independently from 0 to about 3 amino acid residues which are the same or different;
Y is a linker comprising a chain from 0 to about 50 atoms covalently bonded to R₂ and Z when n=0, or to AA₁ and Z when n=1 and m=0, or to AA₂ and Z when n=1 and m=1;
Z is a non-signaling peptide chain that includes a heparin binding domain, comprising an amino acid sequence that comprises (i) a minimum of one heparin binding motif, (ii) a maximum of about ten heparin binding motifs, and (iii) a maximum of about thirty amino acids;
AA₁ and AA₂ are each independently a trifunctional amino acid residue, wherein X and W are covalently bonded through the side chain of AA₁ and AA₂, respectively; and,
n is 0 or 1, and when n is 1, m is 0 or 1, and when n is 0, m is 0.

24. The heparin-binding growth factor analog of claim 23 wherein X, W and Z are synthetic peptide chains.

25. The heparin-binding growth factor analog of claim 23 wherein Y further comprises a linker that (i) is hydrophobic, (ii) comprises a chain of a minimum of about 9 and a maximum of about 50 atoms, and (iii) is not found in the natural ligand of the heparin-binding growth factor receptor (HBGFR) which X binds.

26. The heparin-binding growth factor analog of claim 23 wherein the heparin-binding growth factor analog has an avidity for heparin such that the synthetic heparin-binding growth factor analog binds heparin in 0.15 M NaCl, but is eluted by 1 M NaCl.

27. The heparin-binding growth factor analog of claim 23 wherein the construct is of formula VI: 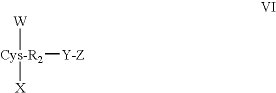
wherein:
Cys is cysteine; and
R₂ is a linker consisting of a sulfhydryl reactive homo-bifunctional cross-linker and a second Cys or comprising a hetero-bifunctional cross-linker.

28. The heparin-binding growth factor analog of claim 27 wherein the construct is of formula VII: 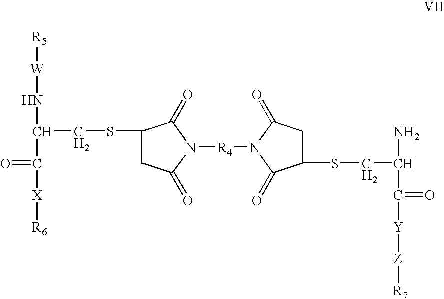
wherein:
R₄ is a linker comprising a chain of between 1 and about 10 backbone atoms selected from carbon, oxygen, sulfur and nitrogen or mixtures thereof;
R₅ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative;
R₆ is OH, NH₂, or NH—R₅; and
R₇ is NH₂, an acyl group with a linear or branched C₁ to C₁₇ alkyl, aryl, heteroaryl, alkene, alkenyl or aralkyl chain including an N-terminus NH₂, NH₃⁺, or NH group or a corresponding acylated derivative.

29. The heparin-binding growth factor analog of claim 28 wherein the construct is of formula VIII: 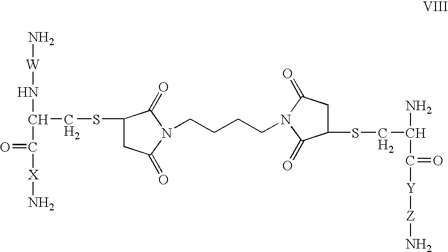
wherein:
X and W are each independently selected from any of SEQ ID NOS:6-55;
Y is between two and about five amino acid residues selected from the group consisting of 6-aminohexanoic acid, 7-aminoheptanoic acid, 9-aminononanoic acid and mixtures thereof; and
Z is any of SEQ ID NOS:2-5 or SEQ ID NO:58.

30. The heparin-binding growth factor analog of claim 23 wherein the covalent bonds between the side chain of R₁ and R₂ and between R₂ and Z when n=0, or between AA₁ and Z when n=1 and m=0, or between AA₂ and Z when n=1 and m=1, comprise an amide, disulfide, thioether, Schiff base, reduced Schiff base, imide, secondary amine, carbonyl, urea, hydrazone or oxime bond.

31. The heparin-binding growth factor analog of claim 1 wherein X and W each independently comprise an amino acid sequence found in any of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, glial derived neurotrophic factor (GDNF), GRO2, hepatoma-derived growth factor (HDGF), and granulocyte-macrophage colony stimulating factor (GMCSF), a homolog of any amino acid sequence found in the foregoing, a reverse sequence of any amino acid sequence found in the foregoing, or a homolog of a reverse sequence of any amino acid residue found in the foregoing.


SEQUENCE LISTING (61 sequences)

SEQ ID NO: 1 - protein, 6 aa, Artificial
  Heparin-binding motif
XXXXXX

SEQ ID NO: 2 - protein, 10 aa, Artificial
  Heparin-binding motif of Z region
RKRKLERIAR

SEQ ID NO: 3 - protein, 10 aa, Artificial
  Heparin-binding motif of Z reqion
RKRKLGRIAR

SEQ ID NO: 4 - protein, 10 aa, Artificial
  Heparin-binding motif of Z region
RKRKLWRARA

SEQ ID NO: 5 - protein, 11 aa, Artificial
  Heparin-binding motif of Z region
RKRKLERIARC

SEQ ID NO: 6 - protein, 15 aa, Artificial
  Synthetic FGF-2 analog
YRSRKYTSWYVALKR

SEQ ID NO: 7 - protein, 28 aa, Artificial
  Synthetic  FGF analog
NRFHSWDCIKTWASDTFVLVCYDDGSEA

SEQ ID NO: 8 - protein, 15 aa, Artificial
  Synthetic FGF-2 analog
HIKLQLQAEERGVVS

SEQ ID NO: 9 - protein, 17 aa, artificial
  Synthetic FGF-1 analog
YISKKHAEKNWFVGLKK

SEQ ID NO: 10 - protein, 15 aa, Artificial
  Synthetic FGF-1 analog
HIQLQLSAESVGEVY

SEQ ID NO: 11 - protein, 20 aa, Artificial
  Synthetic  FGF-7 analog
YASAKWTHNGGEMFVALNQK

SEQ ID NO: 12 - protein, 15 aa, Artificial
  Synthetic FGF-7 analog
YNIMEIRTVAVGIVA

SEQ ID NO: 13 - protein, 20 aa, Artificial
  Synthetic FGF-10 analog
YASFNWQHNGRQMYVALNQK

SEQ ID NO: 14 - protein, 19 aa, Artificial
  Synthetic FGF-22 analog
YASQRWRRRGQPNLALDRR

SEQ ID NO: 15 - protein, 21 aa, Artificial
  Synthetic FGF-9 analog
YSSNLYKHVDTGRRYYVALNK

SEQ ID NO: 16 - protein, 20 aa, Artificial
  Synthetic FGF-16 analog
YASTLYKHSDSERQYVALNK

SEQ ID NO: 17 - protein, 20 aa, Artificial
  Synthetic FGF-20 analog
YSSNIYKHGDTGRRFVALNK

SEQ ID NO: 18 - protein, 16 aa, Artificial
  Synthetic FGF-4 analog
YESYKYPGMFIALSKN

SEQ ID NO: 19 - protein, 17 aa, Artificial
  Synthetic FGF-6 analog
YESDLYQGTYILSKYGR

SEQ ID NO: 20 - protein, 20 aa, Artificial
  Synthetic FGF-12 analog
YSSTLYRQQESGRAWFLGNK

SEQ ID NO: 21 - protein, 21 aa, Artificial
  Synthetic FGF-14 analog
YSSMLYRQQESGRAWFLGLNK

SEQ ID NO: 22 - protein, 21 aa, Artificial
  Synthetic FGF-13 analog
YSSMIYRQQQSGRGWYLGLNK

SEQ ID NO: 23 - protein, 19 aa, Artificial
  Synthetic FGF-11 analog
YASALYRQRRSGRAWYLDK

SEQ ID NO: 24 - protein, 20 aa, Artificial
  Synthetic  VEGF analog
APMAEGGGQNHHEVVKFMDV

SEQ ID NO: 25 - protein, 11 aa, Artificial
  Synthetic VEGF analog
GATWLPPNPTK

SEQ ID NO: 26 - protein, 20 aa, Artificial
  Synthetic VEGF analog
NFLLSWVHWSLALLLYLHHA

SEQ ID NO: 27 - protein, 13 aa, Artificial
  Synthetic BMP  analog
LYVDFSDVGWNDW

SEQ ID NO: 28 - protein, 15 aa, Artificial
  Synthetic BMP analog
AISMLYLDENEKVVL

SEQ ID NO: 29 - protein, 17 aa, Artificial
  Synthetic BMP analog
ISMLYLDENEKVVLKNY

SEQ ID NO: 30 - protein, 15 aa, Artificial
  Synthetic BMP  analog
EKVVLKNYQDMVVEG

SEQ ID NO: 31 - protein, 16 aa, Artificial
  Synthetic BMP  analog
LVVKENEDLYLMSIAC

SEQ ID NO: 32 - protein, 16 aa, Artificial
  Synthetic BMP analog
AFYCHGECPFPLADHL

SEQ ID NO: 33 - protein, 23 aa, Artificial
  Synthetic BMP  analog
PFPLADHLNSTNHAIVQTLVNSV

SEQ ID NO: 34 - protein, 21 aa, Artificial
  Synthetic TGF-beta1  analog
IVYYVGRKPKVEQLSNMIVRS

SEQ ID NO: 35 - protein, 22 aa, Artificial
  Synthetic TGF-beta2 analog
TILYYIGKTPKIEQLSNMIVKS

SEQ ID NO: 36 - protein, 21 aa, Artificial
  Synthetic TGF-beta3  analog
LTILYYVGRTPKVEQLSNMVV

SEQ ID NO: 37 - protein, 23 aa, Artificial
  Synthetic BMP-2 analog
AISMLYLDENEKVVLKNYQDMVV

SEQ ID NO: 38 - protein, 24 aa, Artificial
  Synthetic BMP-3  analog
SSLSILFFDENKNVVLKVYPNMTV

SEQ ID NO: 39 - protein, 24 aa, Artificial
  Synthetic BMP-3beta  analog
NSLGVLFLDENRNVVLKVYPNMSV

SEQ ID NO: 40 - protein, 23 aa, Artificial
  Synthetic BMP-4  peptide analog
AISMLYLDEYDKVVLKNYQEMVV

SEQ ID NO: 41 - protein, 23 aa, Artificial
  Synthetic  BMP-5  analog
AISVLYFDDSSNVILKKYRNMVV

SEQ ID NO: 42 - protein, 23 aa, Artificial
  Synthetic BMP-6  analog
AISVLYFDDNSNVILKKYRNMVV

SEQ ID NO: 43 - protein, 23 aa, Artificial
  Synthetic BMP-7 analog
AISVLYFDDSSNVILKKYRNMVV

SEQ ID NO: 44 - protein, 23 aa, Artificial
  Synthetic BMP-8 analog
ATSVLYYDSSNNVILRKARNMVV

SEQ ID NO: 45 - protein, 23 aa, Artificial
  Synthetic BMP-9 analog
ISVLYKDDMGVPTLKYHYEGMSV

SEQ ID NO: 46 - protein, 22 aa, Artificial
  Synthetic BMP-10 analog
ISILYLDKGVVTYKFKYEGMAV

SEQ ID NO: 47 - protein, 22 aa, Artificial
  synthetic BMP-11 analog
INMLYFNDKQQIIYGKIPGMVV

SEQ ID NO: 48 - protein, 22 aa, Artificial
  Synthetic BMP-12 analog
ISILYIDAANNVVYKQYEDMVV

SEQ ID NO: 49 - protein, 22 aa, Artificial
  Synthetic BMP-13 analog
ISILYIDAGNNVVYKQYEDMVV

SEQ ID NO: 50 - protein, 22 aa, Artificial
  Synthetic BMP-14 analog
ISILFIDSANNVVYKQYEDMVV

SEQ ID NO: 51 - protein, 22 aa, Artificial
  Synthetic BMP-15 analog
ISVLMIEANGSILYKEYEGMIA

SEQ ID NO: 52 - protein, 22 aa, Artificial
  Synthetic GDF-1 analog
ISVLFFDNSDNVVLRQYEDMVV

SEQ ID NO: 53 - protein, 22 aa, Artificial
  Synthetic GDF-3  analog
ISMLYQDNNDNVILRHYEDMVV

SEQ ID NO: 54 - protein, 22 aa, Artificial
  Synthetic GDF-8 analog
INMYLFNGKEQIIYGKIPAMVV

SEQ ID NO: 55 - protein, 22 aa, Artificial
  Synthetic GDF-9 analog
LSVLTIEPDGSIAYKEYEDMIA

SEQ ID NO: 56 - protein, 13 aa, Artificial
  Heparin binding motif
CXXXRKRLDRIAR

SEQ ID NO: 57 - protein, 27 aa, Artificial
  Peptide precursor
LYVDFSDVGWNDWCLYVDFSDVGWNDW

SEQ ID NO: 58 - protein, 9 aa, Artificial
  Heparin binding motif
RKRLDRIAR

SEQ ID NO: 59 - protein, 14 aa, Artificial
  Peptide precursor
CXXXXRKRLDRIAR

SEQ ID NO: 60 - protein, 31 aa, Artificial
  Peptide precursor
AISMLYLDENEKVVLCAISMLYLDENEKVVL

SEQ ID NO: 61 - protein, 14 aa, Artificial
  Peptide precursor
CXXXXRKRLDRIAR